(19) United States
(12) Patent Application Publication
(10) Pub. No.: US 2026/0347840 A1
(43) Pub. Date: Nov. 5, 2026

(54) PROPHYLACTIC AND/OR THERAPEUTIC AGENT FOR HEART DISEASES AND LAMINOPATHIES

(71) Applicant: National Cerebral and Cardiovascular Center, Suita-shi, Osaka (JP)

(72) Inventors: Yasunori SHINTANI, Suita (JP); Yusuke TAKAHASHI, Suita (JP); Issei YAZAWA, Suita (JP)

(73) Assignee: National Cerebral and Cardiovascular Center, Suita-shi, Osaka (JP)

(21) Appl. No.: 19/108,052

(22) PCT Filed: Aug. 28, 2023
(86) PCT No.: PCT/JP2023/031020

§ 371 Date: Feb. 28, 2025

(30) Foreign Application Priority Data
Aug. 29, 2022 (JP) 2022-136279

(51) Int. Cl. C12N 15/113; A61P 9/10

(52) U.S. Cl. CPC C12N 15/113; A61P 9/10; C12N 2310/12; C12N 2310/14

(57) ABSTRACT

The present invention provides a mechanosignaling inhibitor containing a substance inhibiting the expression or function of LSMEM2 as an active ingredient.

### TECHNICAL FIELD

The present invention relates to a mechanosignaling inhibitor, an anti-inflammatory agent, a prophylactic and/or therapeutic agent for heart diseases, a prophylactic and/or therapeutic agent for laminopathies, and the like, which contain a substance inhibiting the expression or function of LSMEM2.

### BACKGROUND ART

In dilated cardiomyopathy (DCM), which is the most frequent causative disease of heart transplant cases in Japan, it is known that the prognosis of a patient group (cardiolaminopathy) having a pathogenic variant in the lamin gene (LMNA) is particularly poor (Non Patent Literature 1). LMNA gene variants are found in about 10% of patients with dilated cardiomyopathy, and cardiolaminopathy is an incurable disease that complicates severe heart failure and fatal arrhythmia with high penetrance (Non Patent Literature 2). In the Cardiomyopathy Clinical Practice Guideline of the Japanese Circulation Society, LMNA gene search is recommended for dilated cardiomyopathy that is familial and complicated with conduction disorder, but a disease-specific therapy for cardiolaminopathy has not been established.

It is known that lamin, which is a lining protein of the nuclear membrane, controls gene expression by propagating extracellular mechanostress to the nucleus via an LINC (Linker of Nucleoskeleton and Cytoskeleton) complex. Since SUN1 KO mice deficient in SUN1, which is a protein constituting the LINC complex, improves the life prognosis of a cardiolaminopathy model mouse (Non Patent Literature 3), it is considered that mechanostress induced from the extracellular to the nucleus is important for the onset of cardiolaminopathy. However, SUN1 is ubiquitous in expression and KO mice are considered to be difficult to target therapeutically because they present infertility and hearing loss.

Recently, it has been reported that the life prognosis of a cardiolaminopathy model mouse is improved by expressing a SUN1 protein having a dominant negative effect of deleting the function of the LINC complex in a cardiomyocyte-specific manner using an AAV vector (Non Patent Literature 4). Although clear side effects were not observed in mice, there remains a concern about adaptation to humans in that the SUN1 KO mice exhibit infertility and hearing loss and destroy the original function of the LINC complex. It is known that p38 MAPK signaling is enhanced in cardiolaminopathy, and it has been reported that a p38a inhibitor improves the cardiac function of a cardiolaminopathy model mouse (Non Patent Literature 5). The p38a inhibitor is currently in phase3 clinical trials for cardiolaminopathy, but KO mice with p38a are embryonic lethal, and there is still a concern about safety for humans.

Therefore, there is a great unmet medical need for the development of an effective therapy for cardiolaminopathy.

### CITATION LIST

### Non Patent Literature

Non Patent Literature 1: Sci Rep. 2018; 8: 1998
Non Patent Literature 2: Eur Heart J. 2018; 39(10): 853
Non Patent Literature 3: Cell. 2012; 149(3): 565.
Non Patent Literature 4: Nat Commun. 2021; 12(1): 4722.
Non Patent Literature 5: Hum Mol Genet. 2012; 21(19): 4325

### SUMMARY OF INVENTION

### Technical Problem

An object of the present invention is to provide a prophylactic and/or therapeutic agent for heart diseases and laminopathies, particularly to provide an effective prophylactic and/or therapeutic agent for cardiolaminopathies.

### Solution to Problem

The present inventors have found that the intercalated disc is a receptor of mechanostress, and have uniquely identified a membrane protein LSMEM2 localized to the intercalated disc and involved in mechanosignaling. LSMEM2 knockout mice exhibited normal cardiac function at rest. The present inventors have found that cardiac function is improved in a mouse generated by crossbreeding a LSMEM2 knockout mouse with a cardiolaminopathy model mouse, thereby completing the present invention. It was also found that LSMEM2 knockout mice suppress immune cell infiltration in a pressure-loaded heart failure model due to coarctation of the aorta, and that lower limb muscle strength is improved in mice generated by crossbreeding with a cardiolaminopathy model mouse, and it was shown that the treatment targeting LSMEM2 can be applied not only as a method for treating cardiolaminopathy but also as a method for treating severe heart failure or laminopathies due to other etiologies.

That is, the present invention is as follows.
[1] A mechanosignaling inhibitor containing a substance inhibiting the expression or function of LSMEM2 as an active ingredient.
[2] The inhibitor according to [1], in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[3] The inhibitor according to [1], in which the substance inhibiting the function of LSMEM2 is an antibody or aptamer against LSMEM2.
[4] The inhibitor according to any one of [1] to [3], in which the substance is provided in a form of one or more expression vectors encoding the substance.
[5] The inhibitor according to any one of [1] to [4], which is an anti-inflammatory agent.
[6] The inhibitor according to any one of [1] to [5], which is a prophylactic and/or therapeutic agent for heart diseases.
[7] The inhibitor according to [6], in which the heart diseases include dilated cardiomyopathy.
[8] An inhibitor of abnormal vesicle trafficking, the inhibitor containing a substance inhibiting the expression or function of LSMEM2 as an active ingredient.
[8-1] A mechanosignaling inhibitor containing a substance inhibiting the expression or function of LSMEM2 as an active ingredient, the inhibitor being an inhibitor of abnormal vesicle trafficking.
[9] A prophylactic and/or therapeutic agent for laminopathies, the agent containing a substance inhibiting the expression or function of LSMEM2 as an active ingredient.
[9-1] A mechanosignaling inhibitor containing a substance inhibiting the expression or function of LSMEM2 as an active ingredient, the inhibitor being a prophylactic and/or therapeutic agent for laminopathies.
[10] The agent according to [9], wherein the laminopathies are selected from muscular dystrophy, cardiolaminopathy, lipodystrophy, or leukodystrophy.
[10-1] A mechanosignaling inhibitor containing a substance inhibiting the expression or function of LSMEM2 as an active ingredient, the inhibitor being a prophylactic and/or therapeutic agent for laminopathies.

In another embodiment, the present invention is as follows.
[A1] A mechanosignaling inhibitor containing a substance inhibiting the expression or function of LSMEM2 as an active ingredient.
[A2] The inhibitor according to [A1], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[A3] The inhibitor according to [A1], in which the substance inhibiting the function of LSMEM2 is an antibody, degrader, or aptamer against LSMEM2.
[A4] The inhibitor according to any one of [A1] to [A3], in which the substance is provided in a form of one or more expression vectors encoding the substance.
[A5] An anti-inflammatory agent, the agent containing a substance inhibiting the expression or function of LSMEM2 as an active ingredient.
[A6] The agent according to [A5], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[A7] The agent according to [A5], in which the substance inhibiting the function of LSMEM2 is an antibody, degrader, or aptamer against LSMEM2.
[A8] The agent according to any one of [A5] to [A7], in which the substance is provided in a form of one or more expression vectors encoding the substance.
[A9] A prophylactic and/or therapeutic agent for heart diseases, the agent containing a substance inhibiting the expression or function of LSMEM2 as an active ingredient.
[A10] The agent according to [A9], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[A11] The agent according to [A9], in which the substance inhibiting the function of LSMEM2 is an antibody, degrader, or aptamer against LSMEM2.
[A12] The agent according to any one of [A9] to [A11], in which the substance is provided in a form of one or more expression vectors encoding the substance.
[A13] The agent according to any one of [A9] to [A12], in which the heart diseases include dilated cardiomyopathy.
[A14] An inhibitor of abnormal vesicle trafficking, the inhibitor containing a substance inhibiting the expression or function of LSMEM2 as an active ingredient.
[A15] The inhibitor according to [A14], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[A16] The inhibitor according to [A14], in which the substance inhibiting the function of LSMEM2 is an antibody, degrader, or aptamer against LSMEM2.
[A17] The inhibitor according to any one of [A14] to [A16], in which the substance is provided in a form of one or more expression vectors encoding the substance.
[A18] A prophylactic and/or therapeutic agent for laminopathies, the agent containing a substance inhibiting the expression or function of LSMEM2 as an active ingredient.
[A19] The agent according to [A18], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[A20] The agent according to [A18], in which the substance inhibiting the function of LSMEM2 is an antibody, degrader, or aptamer against LSMEM2.
[A21] The agent according to any one of [A18] to [A20], in which the substance is provided in a form of one or more expression vectors encoding the substance.
[A22] The agent according to any one of [A18] to [A21], in which the laminopathies are selected from muscular dystrophy, cardiolaminopathy, lipodystrophy, or leukodystrophy.
[B1] A method of inhibiting mechanosignaling in a subject, the method including administering to the subject a substance inhibiting the expression or function of LSMEM2.
[B2] The method according to [B1], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[B3] The method according to [B1], in which the substance inhibiting the function of LSMEM2 is an antibody, degrader, or aptamer against LSMEM2.
[B4] The method according to any one of [B1] to [B3], in which the substance is provided in a form of one or more expression vectors encoding the substance.
[B5] A method of inhibiting inflammation in a subject, the method including administering to the subject a substance inhibiting the expression or function of LSMEM2.
[B6] The method according to [B5], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[B7] The method according to [B5], in which the substance inhibiting the function of LSMEM2 is an antibody, degrader, or aptamer against LSMEM2.
[B8] The method according to any one of [B5] to [B7], in which the substance is provided in a form of one or more expression vectors encoding the substance.
[B9] A method of preventing and/or treating heart diseases in a subject, the method including administering to the subject a substance inhibiting the expression or function of LSMEM2.
[B10] The method according to [B9], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[B11] The method according to [B9], in which the substance inhibiting the function of LSMEM2 is an antibody, degrader, or aptamer against LSMEM2.
[B12] The method according to any one of [B9] to [B11], in which the substance is provided in a form of one or more expression vectors encoding the substance.
[B13] The method according to any one of [B9] to [B12], in which the heart diseases include dilated cardiomyopathy.
[B14] A method for inhibiting abnormal vesicle trafficking in a subject, the method including administering to the subject a substance inhibiting the expression or function of LSMEM2.
[B15] The method according to [B14], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[B16] The method according to [B14], in which the substance inhibiting the function of LSMEM2 is an antibody, degrader, or aptamer against LSMEM2.
[B17] The method according to any one of [B14] to [B16], in which the substance is provided in a form of one or more expression vectors encoding the substance.
[B18] A method of preventing and/or treating laminopathies, the method including administering to a subject a substance inhibiting the expression or function of LSMEM2.
[B19] The method according to [B18], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[B20] The method according to [B18], in which the substance inhibiting the function of LSMEM2 is an antibody, degrader, or aptamer against LSMEM2.
[B21] The method according to any one of [B18] to [B20], in which the substance is provided in a form of one or more expression vectors encoding the substance.
[B22] The method according to any one of [B18] to [B21], in which the laminopathies are selected from muscular dystrophy, cardiolaminopathy, lipodystrophy, or leukodystrophy.
[C1] A composition for use in inhibition of mechanosignaling, the composition containing a substance inhibiting the expression or function of LSMEM2.
[C2] The composition according to [C1], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[C3] The composition according to [C1], in which the substance inhibiting the function of LSMEM2 is an antibody, degrader, or aptamer against LSMEM2.
[C4] The composition according to any one of [C1] to [C3], in which the substance is provided in a form of one or more expression vectors encoding the substance.
[C5] A composition for use in inhibition of inflammation, the composition containing a substance inhibiting the expression or function of LSMEM2.
[C6] The composition according to [C5], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[C7] The composition according to [C5], in which the substance inhibiting the function of LSMEM2 is an antibody, degrader, or aptamer against LSMEM2.
[C8] The composition according to any one of [C5] to [C7], in which the substance is provided in a form of one or more expression vectors encoding the substance.
[C9] A composition for use in prevention and/or treatment of heart diseases, the composition containing a substance inhibiting the expression or function of LSMEM2.
[C10] The composition according to [C9], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[C11] The composition according to [C9], in which the substance inhibiting the function of LSMEM2 is an antibody, degrader, or aptamer against LSMEM2.
[C12] The composition according to any one of [C9] to [C11], in which the substance is provided in a form of one or more expression vectors encoding the substance.
[C13] The composition according to any one of [C9] to [C12], in which the heart diseases include dilated cardiomyopathy.
[C14] A composition for use in inhibition of abnormal vesicle trafficking, the composition containing a substance inhibiting the expression or function of LSMEM2.
[C15] The composition according to [C14], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[C16] The composition according to [C14], in which the substance inhibiting the function of LSMEM2 is an antibody, degrader, or aptamer against LSMEM2.
[C17] The composition according to any one of [C14] to [C16], in which the substance is provided in a form of one or more expression vectors encoding the substance.
[C18] A composition for use in prevention and/or treatment of laminopathies, the composition containing a substance inhibiting the expression or function of LSMEM2.
[C19] The composition according to [C18], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[C20] The composition according to [C18], in which the substance inhibiting the function of LSMEM2 is an antibody, degrader, or aptamer against LSMEM2.
[C21] The composition according to any one of [C18] to [C20], in which the substance is provided in a form of one or more expression vectors encoding the substance.
[C22] The composition according to any one of [C18] to [C21], in which the laminopathies are selected from muscular dystrophy, cardiolaminopathy, lipodystrophy, or leukodystrophy.
[D1] Use of a substance inhibiting the expression or function of LSMEM2 in production of a medicament for inhibiting mechanosignaling.
[D2] The use according to [D1], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[D3] The use according to [D1], in which the substance inhibiting the function of LSMEM2 is an antibody, degrader, or aptamer against LSMEM2.
[D4] The use according to any one of [D1] to [D3], in which the substance is provided in a form of one or more expression vectors encoding the substance.
[D5] Use of a substance inhibiting the expression or function of LSMEM2 in production of a medicament for inhibiting inflammation.
[D6] The use according to [D5], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[D7] The use according to [D5], in which the substance inhibiting the function of LSMEM2 is an antibody, degrader, or aptamer against LSMEM2.
[D8] The use according to any one of [D5] to [D7], in which the substance is provided in a form of one or more expression vectors encoding the substance.
[D9] Use of a substance inhibiting the expression or function of LSMEM2 in production of a medicament for preventing and/or treating heart diseases.
[D10] The use according to [D9], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[D11] The use according to [D9], in which the substance inhibiting the function of LSMEM2 is an antibody, degrader, or aptamer against LSMEM2.
[D12] The use according to any one of [D9] to [D11], in which the substance is provided in a form of one or more expression vectors encoding the substance.
[D13] The use according to any one of [D9] to [D12], in which the heart diseases include dilated cardiomyopathy.
[D14] Use of a substance inhibiting the expression or function of LSMEM2 in production of a medicament for inhibiting abnormal vesicle trafficking.
[D15] The use according to [D14], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[D16] The use according to [D14], in which the substance inhibiting the function of LSMEM2 is an antibody, degrader, or aptamer against LSMEM2.
[D17] The use according to any one of [D14] to [D16], in which the substance is provided in a form of one or more expression vectors encoding the substance.
[D18] Use of a substance inhibiting the expression or function of LSMEM2 in production of a medicament for preventing and/or treating laminopathies.
[D19] The use according to [D18], in which
the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.
[D20] The use according to [D18], in which the substance inhibiting the function of LSMEM2 is an antibody, degrader, or aptamer against LSMEM2.
[D21] The use according to any one of [D18] to [D20], in which the substance is provided in a form of one or more expression vectors encoding the substance.
[D22] The use according to any one of [D18] to [D21], in which the laminopathies are selected from muscular dystrophy, cardiolaminopathy, lipodystrophy, or leukodystrophy.

### Advantageous Effects of Invention

According to the present invention, a mechanosignaling inhibitor containing a substance inhibiting the expression or function of LSMEM2 can be provided, so that it is possible to effectively treat cardiolaminopathy, severe heart failure due to other etiologies, and laminopathies.

### BRIEF DESCRIPTION OF DRAWINGS

FIG. 1 is a view showing organ specificity of expression of LSMEM2 by RT-PCR.

FIG. 2 is a view showing the results of RNA-seq of LSMEM2 overexpressing cardiomyocytes.

FIG. 3 is a view showing that LSMEM2 is essential for mechanostress-dependent (mechanical stretch) inflammatory signals.

FIG. 4 is a view showing production and analysis of LSMEM2 knockout mice.

FIG. 5 is a view showing that LSMEM2 KO improves the survival rate, cardiac function, cardiac inflammation signal, and skeletal muscle strength of a laminopathy model (LMNA H222P KI/KI).

FIG. 6 is a view showing a cardiac function improving effect of knockdown by MyoAAV2 shRNA LSMEM2.

FIG. 7 is a view showing analysis of LSMEM2 knockout in a pressure-loaded heart failure model.

### DESCRIPTION OF EMBODIMENTS

LSMEM2, which is a target molecule of a mechanosignaling inhibitor of the present invention, is a single transmembrane protein localized in the intercalated disc, which is a receptor of mechanostress, and in humans, isoform 1 (NP_694947.1) and isoform 2 (NP 001291314.1) exist, in which isoform 1 has an amino acid sequence composed of 164 amino acids represented by SEQ ID NO: 2 translated from the mRNA represented by SEQ ID NO: 1, and among them, 1 to 99th positions are intracellular regions, 100 to 122nd positions are transmembrane regions, and 123 to 164th positions are extracellular regions. Serine at position 50 is a phosphorylation site and asparagine at position 156 is a glycosylation site. isoform 2 (SEQ ID NO: 4) translated from the mRNA represented by SEQ ID NO: 3 is shorter by one amino acid than isoform 1 and consists of 163 amino acids.

In the present specification, “LSMEM2” is a protein including an amino acid sequence that is the same as or substantially the same as the amino acid sequence represented by SEQ ID NO: 2 (isoform 1) or SEQ ID NO: 4 (isoform 2). In the present specification, proteins and peptides are described with an N-terminus at the left end (amino-terminus) and a C-terminus at the right end (carboxyl-terminus) according to the peptide labeling convention.

The “amino acid sequence that is the same as or substantially the same as the amino acid sequence represented by SEQ ID NO: 2 or SEQ ID NO: 4” means
(a) an amino acid sequence of ortholog of human LSMEM2 consisting of the amino acid sequence represented by SEQ ID NO: 2 or SEQ ID NO: 4 in other warm-blooded animals (such as guinea pig, rat, mouse, chicken, rabbit, dog, pig, sheep, cow, and monkey); or
(b) an amino acid sequence in a natural allelic mutant or genetic polymorphism of human LSMEM2 or the ortholog of the above (a) consisting of the amino acid sequence represented by SEQ ID NO: 2 or SEQ ID NO: 4.

Preferably, LSMEM2 is human LSMEM2 consisting of the amino acid sequence represented by SEQ ID NO: 2 or SEQ ID NO: 4, or a natural allelic mutant or genetic polymorphism thereof.

LSMEM2 KO mice develop normally, have completely normal cardiac function, and no problems with reproductive function. That is, inhibiting of any of mRNA, protein, and downstream signals without limitation of modalities such as gene therapy, genome editing, an antibody medicament, a nucleic acid medicament (antisense, siRNA, miRNA, aptamer, and the like, but not limited thereto), and a low-molecular-weight compound for LSMEM2 is a promising drug discovery target as a novel therapeutic method.

One aspect of the present invention is a mechanosignaling inhibitor containing a substance inhibiting the expression or function of LSMEM2 as an active ingredient. The “substance inhibiting the expression of LSMEM2” in the present invention may be a substance that edits the LSMEM2 gene at a genomic level, or may act at any stage such as a transcription level, a level of post-transcriptional regulation, a level of translation into a protein, or a level of post-translational modification. Therefore, examples of the substance inhibiting the expression of LSMEM2 include a substance inhibiting transcription of an LSMEM2 gene (e.g., antigene), a substance inhibiting processing from an initial transcription product to mRNA, a substance inhibiting transport of mRNA to cytoplasm, a substance inhibiting translation from mRNA to protein (e.g., antisense nucleic acid, or miRNA) or degrading mRNA (e.g., siRNA, ribozyme, or miRNA), a substance inhibiting post-translational modification of an initial translation product, and a substance inducing degradation of LSMEM2. A substance acting at any stage can be used, but a substance complementarily binding to mRNA and inhibiting translation into protein or degrading mRNA is preferable.

The substance specifically inhibiting translation of an LSMEM2 gene from mRNA to protein (or degrading mRNA) preferably includes a nucleic acid including a nucleotide sequence complementary to the nucleotide sequence of the mRNA or a part thereof.

The nucleotide sequence complementary to the nucleotide sequence of the mRNA of an LSMEM2 gene means a nucleotide sequence having such complementarity that the nucleotide sequence can bind to a target sequence of the mRNA and inhibit translation thereof (or cleave the target sequence) under physiological conditions, and specifically, for example, the nucleotide sequence is a nucleotide sequence having a homology of 90% or more, preferably 95% or more, more preferably 97% or more, and particularly preferably 98% or more with respect to an overlapping region with a nucleotide sequence completely complementary to the nucleotide sequence of the mRNA (that is, the nucleotide sequence of complementary strand of mRNA). The “homology of nucleotide sequences” in the present invention can be calculated using homology calculation algorithm NCBI BLAST (National Center for Biotechnology Information Basic Local Alignment Search Tool) under the following conditions (expected value=10; allowing a gap; filtering=ON; match score=1; mismatch score=−3).

More specifically, the nucleotide sequence complementary to the nucleotide sequence of the mRNA of an LSMEM2 gene is a nucleotide sequence that hybridizes with the nucleotide sequence represented by SEQ ID NO: 1 or SEQ ID NO: 3 under stringent conditions. Here, examples of the “stringent conditions” include conditions described in Current Protocols in Molecular Biology, John Wiley & Sons, 6.3.1-6.3.6, 1999, for example, hybridization at 6×SSC (sodium chloride/sodium citrate)/45 °C, and then one or more washes at 0.2×SSC/0.1% SDS/50 to 65 °C, and the like, but a person skilled in the art can appropriately select hybridization conditions that impart stringency equivalent thereto.

Preferred examples of the mRNA of the LSMEM2 gene include human LSMEM2 (RefSeq Accession No. NM 153215.3 or NM_001304385.2) including the nucleotide sequence represented by SEQ ID NO: 1 or SEQ ID NO: 3, orthologs thereof in other warm-blooded animals, and mRNA of a natural allelic mutant or genetic polymorphism thereof.

The “part of the nucleotide sequence complementary to the nucleotide sequence of the mRNA of an LSMEM2 gene” is not particularly limited in length and position as long as it can specifically bind to the mRNA of the LSMEM2 gene and can inhibit translation of a protein from the mRNA (or degrade the mRNA), but from the viewpoint of sequence specificity, the part of the nucleotide sequence complementary to the target sequence includes at least 10 bases or more, preferably 15 bases or more, and more preferably 19 bases or more.

Specifically, as the nucleic acid including a part of the nucleotide sequence complementary to the nucleotide sequence of mRNA of an LSMEM2 gene, the following (a) to (c) are preferably exemplified:
(a) a nucleic acid having RNAi activity against mRNA of an LSMEM2 gene or a precursor thereof;
(b) an antisense nucleic acid to mRNA of an LSMEM2 gene; or
(c) a ribozyme nucleic acid to mRNA of an LSMEM2 gene.

The “substance inhibiting the expression of LSMEM2” in the present invention may be a substance inhibiting the expression of LSMEM2 by, for example, modifying the sequence of the LSMEM2 gene in the genome by a genome editing technique. As such a substance, the following substance (d) is preferably exemplified:
(d) a nucleic acid sequence-recognizing module specifically binding to a target region in an LSMEM2 gene.

The substance inhibiting the expression of LSMEM2 in the present invention is not limited to the nucleic acid or nucleic acid sequence-recognizing module including the nucleotide sequence complementary to the nucleotide sequence of the mRNA of an LSMEM2 gene or a part thereof as described above, and may be another substance such as a low-molecular-weight compound, a peptide, a cyclic peptide, a protein, a middle molecule, or a polymer as long as it directly or indirectly inhibits the production of an LSMEM2 protein.

The substance inhibiting the expression of LSMEM2 in the present invention is not limited to the antisense nucleic acid, siRNA, ribozyme, and the like as described above, and may be other substances (low-molecular-weight compound, peptide, cyclic peptide, protein, medium molecule, polymer, and the like) as long as the substance directly or indirectly inhibits the expression of LSMEM2. Examples of such a low-molecular-weight compound include a low-molecular-weight phosphorylation inhibitor that inhibits phosphorylation of serine at position 50 of LSMEM2.

Specific aspects of (a) to (d) described above will be described below.

(a) Nucleic Acid Having RNAi Activity Against mRNA of LSMEM2 Gene or Precursor Thereof

In the present specification, a double-stranded RNA composed of an oligo RNA complementary to the mRNA of an LSMEM2 gene and its complementary strand, so-called siRNA, is defined as being included in the nucleic acid including the nucleotide sequence complementary to the nucleotide sequence of the mRNA of an LSMEM2 gene or a part thereof.

siRNA can be designed based on cDNA sequence information of the target gene, for example, according to the rule proposed by Elbashir et al. (Genes Dev., 15, 188-200 (2001)). Examples of the target sequence of the siRNA include, but are not limited to, AA₊(N)₁₉, AA₊(N)₂₁, and NA₊(N)₂₁ (N is any base). The position of the target sequence is also not particularly limited. For the candidate group of the selected target sequences, whether there is no homology in sequences of 16-17 bases in succession in mRNA other than the target is examined using homology search software such as BLAST so as to confirm the specificity of the selected target sequences. For example, when AA₊(N)₁₉, AA₊(N)₂₁, or NA₊(N)₂₁ (N is any base) is used as a target sequence, a double-stranded RNA including a sense strand having a 3′ terminal overhang of TT or UU at 19-21 bases after AA (or NA) and an antisense strand having a sequence complementary to 19-21 bases and a 3′ terminal overhang of TT or UU may be designed as siRNA for the target sequence of which specificity is confirmed. A short hairpin RNA (shRNA) which is a precursor of siRNA can be designed by appropriately selecting an arbitrary linker sequence (for example, about 5-25 bases) capable of forming a loop structure and linking the sense strand and the antisense strand via the linker sequence.

The sequence of siRNA and/or shRNA can be searched using search software provided on various websites for free. Examples of such a site include, but are not limited to, siDESIGN Center provided by Dharmacon and siRNA Target Finder provided by GenScript. The fact that siRNA or shRNA synthesized based on the hit siRNA sequence information can actually inhibit LSMEM2 expression in cardiomyocytes to a therapeutically effective extent can be verified, for example, by measuring the expression level of LSMEM2 mRNA in cardiomyocyte stem cells into which the nucleic acid has been introduced. When the effect of inhibiting the expression of LSMEM2 mRNA is attenuated after introduction although it originally has RNAi activity, it is possible to further maintain the effect of inhibiting the expression of LSMEM2 by improving the in vivo stability by adding various modifications to the constituent nucleotides of the siRNA or shRNA as described later, and a desired therapeutic effect can be exhibited.

In a preferred embodiment, the siRNA and shRNA of the present invention include a nucleotide sequence complementary to a sequence consisting of at least 15 consecutive nucleotides in a region set forth in nucleotide Nos. 1 to 1434 in the nucleotide sequence represented by SEQ ID NO: 1 or SEQ ID NO: 3. In a particularly preferred embodiment, the siRNA of the present invention is Silencer (registered trademark) Select siRNA (LSMEM2 siRNA ID: s189327, sense (5′-3′): GCACAGUGGAGGCUCACUATT (SEQ ID NO: 9), or antisense (5′-3′): UAGUGAGCCUCCACUGUGCAG (SEQ ID NO: 10) (Thermo Fisher Scientific, Waltham, MA), and the shRNA is 5′-CTCTCACTGATGGCTTCATTT-3′ (SEQ ID NO: 11).

In the present specification, a microRNA (miRNA) targeting the mRNA of an LSMEM2 gene is also defined as being included in the nucleic acid including the nucleotide sequence complementary to the nucleotide sequence of the mRNA of an LSMEM2 gene or a part thereof. The miRNA is involved in post-transcriptional control of gene expression by complementarily binding to a target mRNA to inhibit translation of the mRNA or by degrading the mRNA.

The miRNA is first transcribed from the gene encoding the miRNA by primary-microRNA (pri-miRNA) which is a primary transcription product, then processed by Drosha into about 70 base-long precursor-microRNA (pre-miRNA) having a characteristic hairpin structure, and then transported from the nucleus to the cytoplasm, further converted into a mature miRNA by processing via Dicer, and incorporated into RISC to act on the target mRNA. Therefore, as a precursor of the miRNA, a pre-miRNA or a pri-miRNA, preferably a pre-miRNA can also be used.

The miRNA can be searched using target prediction software provided on various websites for free. Examples of such a site include, but are not limited to, TargetScan published by Whitehead Institute of the United States and DIANA-micro-T-CDS published by Alexander-Fleming Center for Biophysical Sciences in Greek. Alternatively, the miRNA targeting LSMEM2 mRNA can also be searched using TarBase, which is a database on miRNAs experimentally proven to act on target mRNA, published by University of Tessari and Institut Pasteur, and the like. Sequence information of these miRNA and/or pre-miRNA can be obtained using, for example, miRBase published by University of Manchester in the UK.

The nucleotide molecule constituting the siRNA and/or the shRNA or the miRNA and/or the pre-miRNA may be a naturally occurring RNA or DNA, but may include various chemical modifications in order to improve stability (chemical and/or enzyme) and specific activity (affinity with RNA). For example, in order to prevent degradation by a hydrolase such as a nuclease, the phosphate residue (phosphate) of each nucleotide constituting the antisense nucleic acid can be substituted with a chemically modified phosphate residue such as phosphorothioate (PS), methyl phosphonate, or phosphorodithionate. The hydroxyl group at the 2′-position of the sugar (ribose) of each nucleotide may be substituted with —OR (R=CH₃(2′-O-Me), CH₂CH₂OCH₃ (2′-O-MOE), CH₂CH₂NHC(NH)NH₂, CH₂CONHCH₃, CH₂CH₂CN, etc.). The base moiety (pyrimidine, purine) may be chemically modified, and examples thereof include introduction of a methyl group or a cationic functional group to the 5-position of the pyrimidine base, and substitution of the carbonyl group at the 2-position with thiocarbonyl.

Two conformations of sugar moieties of RNA, C2′-endo (S type) and C3′-endo (N type), are dominant, and in single-stranded RNA, these two conformations exist as an equilibrium therebetween, but when a double strand is formed, they are fixed to N type. Therefore, in order to impart strong binding ability to the target RNA, BNA (LNA) (Imanishi, T. et al., Chem. Commun., 1653-9, 2002; Jepsen, J. S. et al., Oligonucleotides, 14, 130-46, 2004) or ENA (Morita, K. et al., Nucleosides Nucleotides Nucleic Acids, 22, 1619-21, 2003), which is an RNA derivative in which the conformation of the sugar moiety is fixed to N type by crosslinking 2′oxygen and 4′carbon, can also be preferably used.

The siRNA can be prepared by synthesizing a sense strand and an antisense strand of a target sequence on mRNA with a DNA/RNA automatic synthesizer, respectively, denaturing the sense strand and the antisense strand at about 90 to about 95 °C for about 1 minute in an appropriate annealing buffer, and then annealing the sense strand and the antisense strand at about 30 to about 70 °C for about 1 to about 8 hours. The siRNA can also be prepared by synthesizing shRNA to be a precursor of siRNA and cleaving it using dicer. The miRNA and the pre-miRNA can be synthesized by a DNA/RNA automatic synthesizer based on their sequence information.

In the present specification, a nucleic acid designed to be able to generate siRNA or miRNA against the mRNA of an LSMEM2 gene in vivo is also defined as being included in the nucleic acid including the nucleotide sequence complementary to the nucleotide sequence of the mRNA of an LSMEM2 gene or a part thereof. Examples of such a nucleic acid include an expression vector constructed to express the shRNA or siRNA, or miRNA or pre-miRNA described above. The shRNA can be prepared by designing an oligo RNA including a nucleotide sequence in which a sense strand and an antisense strand of a target sequence on mRNA are linked by inserting a spacer sequence having a length (for example, about 5 to 25 bases) capable of forming an appropriate loop structure therebetween, and synthesizing the designed oligo RNA with a DNA/RNA automatic synthesizer. The vector expressing the shRNA includes a tandem type and a stem-loop (hairpin) type. In the former, an expression cassette of a sense strand and an expression cassette of an antisense strand of siRNA are linked in tandem, and each strand is expressed in a cell and annealed to form a double-stranded siRNA (dsRNA). On the other hand, in the latter, an expression cassette of shRNA is inserted into a vector, and shRNA is expressed in a cell and processed by dicer to form dsRNA. As the promoter, a polII promoter (for example, a CMV immediate early promoter) can also be used, but it is common to use a polIII promoter in order to accurately transcribe a short RNA. Examples of the polIII promoter include mouse and human U6-snRNA promoters, human H1-RNase P RNA promoters, and human valine-tRNA promoters. A sequence in which four or more Ts are continuous is used as a transcription termination signal. The expression cassette of the miRNA or the pre-miRNA can also be produced in the same manner as the shRNA.

The siRNA or shRNA, or miRNA or pre-miRNA expression cassette thus constructed is then inserted into a plasmid vector or a viral vector. As such a vector, a viral vector such as retrovirus, lentivirus, adenovirus, adeno-associated virus, herpesvirus, or Sendai virus, an animal cell expression plasmid, or the like is used.

For example, in the case of preparing an adeno-associated virus (AAV) vector, first, the ITRs at both ends of the genomic sequence of the wild-type AAV are left, and a vector plasmid into which a desired nucleic acid is inserted is prepared instead of DNA encoding the other Rep protein and capsid protein. On the other hand, DNA encoding Rep protein and capsid protein required for formation of virus particles is inserted into another plasmid. A plasmid containing genes (E1A, E1B, E2A, VA, and E4orf6) responsible for the helper action of adenovirus necessary for proliferation of AAV is prepared as an adenovirus helper plasmid. Co-transfection of these three plasmids into a host cell results in the production of recombinant AAV (that is, AAV vector) in the cell. As the host cell, a cell capable of supplying a part of the gene products (proteins) of the genes responsible for the helper action (for example, 293 cell or the like) is preferably used, and in the case of using such a cell, it is not necessary to carry the gene encoding a protein that can be supplied from the host cell on the adenovirus helper plasmid. Since the produced AAV vector is present in the nucleus, a desired AAV vector is prepared by freeze-thawing and collecting the host cell, and performing separation and purification by a density gradient ultracentrifugation method using cesium chloride, a column method, or the like.

(b) Antisense Nucleic Acid to mRNA of LSMEM2 Gene

The “antisense nucleic acid to mRNA of an LSMEM2 gene” in the present invention is a nucleic acid including the nucleotide sequence complementary to the nucleotide sequence of the mRNA or a part thereof, and has a function of inhibiting protein synthesis by forming and binding a specific and stable duplex with a target mRNA.

Examples of the antisense nucleic acid include polydeoxyribonucleotides containing 2-deoxy-D-ribose, polyribonucleotides containing D-ribose, other types of polynucleotides that are N-glycosides of purine or pyrimidine bases, other polymers having non-nucleotide backbones (for example, commercially available protein nucleic acids and synthetic sequence specific nucleic acid polymers), and other polymers containing special bonds (provided that the polymers contain a nucleotide having an arrangement allowing base pairing and base attachment as found in DNA or RNA). These may be double-stranded DNA, single-stranded DNA, double-stranded RNA, single-stranded RNA, or DNA:RNA hybrids, and further may be unmodified polynucleotides (or unmodified oligonucleotides), ones added with known modification, for example, labeled ones known in the art, ones with a cap, methylated one, one or more natural nucleotides substituted by an analog, ones with intramolecular nucleotide modification, for example, ones with non-charge bond (for example, methylphosphonate, phosphotriester, phosphoramidate, carbamate, and the like), ones with a charged bond or sulfur-containing bond (e.g., phosphorothioate, phosphorodithioate, and the like), for example, ones with a side chain group such as a protein (e.g., nuclease, nuclease inhibitor, toxin, antibody, signal peptide, poly-L-lysine, or the like) or sugar (e.g., monosaccharide or the like), ones with an intercalating compound (e.g., acridine, psoralen, or the like), ones containing a chelate compound (for example, a metal, a radioactive metal, boron, an oxidative metal, or the like), ones containing an alkylating agent, or ones with a modified bond (for example, a anomer-type nucleic acid, or the like). Here, the “nucleoside”, “nucleotide”, and “nucleic acid” not only contain a purine and a pyrimidine base, but also may contain one having another modified heterocyclic base. Such modifications may include methylated purines and pyrimidines, acylated purines and pyrimidines, or other heterocycles. The modified nucleoside and modified nucleotide may also have a modified sugar moiety, for example, one or more hydroxyl groups may be substituted with a halogen, an aliphatic group, or the like, or may be converted into a functional group such as an ether or an amine.

As described above, the antisense nucleic acid may be DNA or RNA, or may be a DNA/RNA chimera. When the antisense nucleic acid is DNA, the RNA:DNA hybrid formed by the target RNA and the antisense DNA can be recognized by the endogenous RNase H to cause selective degradation of the target RNA. Therefore, in the case of the antisense DNA directed to degradation by RNase H, the target sequence may be not only a sequence in mRNA, but also a sequence of an intron region in an early translation product of the LSMEM2 gene. The intron sequence can be determined by comparing the genomic sequence with the cDNA nucleotide sequence of the LSMEM2 gene using a homology search program such as BLAST or FASTA.

The length of the target region of the antisense nucleic acid of the present invention is not particularly limited as long as translation into a protein is inhibited as a result of hybridization of the antisense nucleic acid, and the target region may be the entire sequence of mRNA encoding a protein or a partial sequence, and examples thereof include the entire sequence of mRNA or an initial transcription product which has a length of about 10 bases for the shortest. In consideration of ease of synthesis, antigenicity, problems of intracellular migration, and the like, an oligonucleotide composed of about 10 to about 40 bases, particularly about 15 to about 30 bases is preferable, but the oligonucleotide is not limited thereto. Specifically, 5′ end hairpin loop, 5′ end 6-base-pair repeats, 5′ end untranslated region, translation initiation codon, protein coding region, ORF translation stop codon, 3′ end untranslated region, 3′ end palindrome region, or 3′ end hairpin loop of the LSMEM2 gene, and the like may be selected as a preferred target region of the antisense nucleic acid, but the target region is not limited thereto.

In one embodiment, as in the siRNA, the target region of the antisense nucleic acid of the present invention can include a sequence consisting of at least 15 consecutive nucleotides in a region set forth in nucleotide Nos. 1 to 1434 in the nucleotide sequence represented by SEQ ID NO: 1 or SEQ ID NO: 3.

The antisense nucleic acid of the present invention may be one (antigene) that hybridizes with mRNA or an initial transcription product of the LSMEM2 gene to inhibit translation into a protein, and may bind to these genes that are double-stranded DNA to form a triple strand (triplex) to inhibit transcription into RNA.

The nucleotide molecule constituting the antisense nucleic acid may also be modified in the same manner as in the case of the above-described siRNA or the like in order to improve stability, specific activity, and the like.

The antisense oligonucleotide of the present invention can be prepared by determining a target sequence of mRNA or an initial transcription product based on a cDNA sequence or a genomic DNA sequence of the LSMEM2 gene, and synthesizing a sequence complementary thereto using a commercially available DNA/RNA automatic synthesizer ((Applied Biosystems, Beckman, etc.). Any of the antisense nucleic acid including various modifications described above can also be chemically synthesized by a technique known per se.

(c) Ribozyme Nucleic Acid to mRNA of LSMEM2 Gene

Other examples of the nucleic acid including the nucleotide sequence complementary to the nucleotide sequence of the mRNA of an LSMEM2 gene or a part thereof include a ribozyme nucleic acid capable of specifically cleaving the mRNA inside the coding region. The “ribozyme” refers to an RNA having an enzyme activity to cleave a nucleic acid in the narrow sense, but in the present specification, is used as a concept encompassing a DNA as long as it has a sequence-specific nucleic acid cleavage activity. As the most versatile ribozyme nucleic acid, there is a self-splicing RNA found in infectious RNAs such as viroid and virusoid, and the hammerhead type, the hairpin type, and the like are known. The hammerhead type exhibits an enzyme activity with about 40 bases in length, and it is possible to specifically cleave the target mRNA by making several bases at both ends adjacent to the hammerhead structure portion (about 10 bases in total) be a sequence complementary to the desired cleavage site of the mRNA. Since this type of ribozyme nucleic acid uses only RNA as a substrate, it has a further advantage that it does not attack genomic DNA. When the mRNA of the LSMEM2 gene itself has a double-stranded structure, the target sequence can be made single-stranded by using a hybrid ribozyme in which an RNA motif derived from a viral nucleic acid capable of specifically binding to an RNA helicase is linked [Proc. Natl. Acad. Sci. USA, 98(10): 5572-5577 (2001)]. When the ribozyme is used in the form of an expression vector containing a DNA encoding the ribozyme, a hybrid ribozyme in which a tRNA modified sequence is further linked can be used in order to promote translocation of a transcription product to cytoplasm [Nucleic Acids Res., 29(13): 2780-2788 (2001)].

The nucleic acid including the nucleotide sequence complementary to the nucleotide sequence of the mRNA of an LSMEM2 gene or a part thereof may be provided in a special form such as a liposome or a microsphere, applied to gene therapy, or provided in an added form. Examples of those used in the added form include polycations such as polylysine that act as charge neutralizers of the phosphate backbone, and or hydrophobic ones such as lipids (e.g., phospholipids, cholesterols, etc.) that enhance the interaction with cell membranes or increase uptake of the nucleic acid. Preferred examples of the lipids to be added include cholesterols or derivatives thereof (e.g., cholesteryl chloroformate, cholic acid, etc.). These may be attached to the 3′ end or the 5′ end of the nucleic acid, and may also be attached thereto through a base, sugar, or intramolecular nucleoside linkage. Examples of the other group include a capping group specifically disposed at the 3′ end or the 5′ end of the nucleic acid for preventing degradation by a nuclease such as exonuclease or RNase. Examples of such a capping group include, but are not limited to, hydroxyl protecting groups known in the art, including glycols such as polyethylene glycol and tetraethylene glycol.

The LSMEM2 protein expression inhibitory activity of these nucleic acids can be examined using a transformant into which the LSMEM2 gene is introduced, an in vivo or in vitro LSMEM2 gene expression system, or an in vivo or in vitro LSMEM2 protein translation system.

### (d) Nucleic Acid Sequence-Recognizing Module Specifically Binding to Target Region in LSMEM2 Gene

(d-1) Substance without Modification of Genomic Sequence

As a conventional genome editing method, a method utilizing an artificial nuclease in which a molecule having a sequence-independent DNA cleavage ability and a molecule having a sequence recognition ability in combination has been well known. For example, there have been reported methods and the like by utilizing a zinc finger nuclease (ZFN) in which a zinc finger DNA binding domain and a non-specific DNA cleavage domain are linked, TALEN in which a transcription activator-like (TAL) effector which is a DNA binding module that the plant pathogenic bacteria Xanthomonas has, and a DNA endonuclease are linked, or CRISPR-Cas9 system in which nucleic acid sequence CRISPR (Clustered Regularly Interspaced Short Palindromic Repeats) that functions in an acquired immune system possessed by eubacterium and archaebacterium, and nuclease Cas (CRISPR-associated) protein family having an important function along with CRISPR are combined. An artificial nuclease in which a nuclease is linked to a PPR protein configured to recognize a specific nucleotide sequence by continuation of a PPR motif consisting of amino acids and recognizing one nucleic acid base has also been reported.

In any of these genome editing techniques, a specific target nucleotide sequence is recognized by a protein (ZF, TAL effector, PPR) or a complex (CRISPR-Cas9) of RNA and a protein. Therefore, by binding to a target region in the LSMEM2 gene using these substances (nucleic acid sequence-recognizing module) that impart sequence specificity to an artificial nuclease, expression of LSMEM2 can be inhibited.

The “nucleic acid sequence-recognizing module” in the present invention means a molecule or a molecular complex having an ability to specifically recognize and bind to a specific nucleotide sequence (that is, target nucleotide sequence) on a DNA strand. Binding of the nucleic acid sequence-recognizing module to the target nucleotide sequence allows the effector linked to the module to specifically act on the targeted site of DNA.

In one aspect of the present invention, as the nucleic acid sequence-recognizing module, CRISPR-Cas system is exemplified. Since the CRISPR-Cas system recognizes a sequence of a target DNA by a complex of a short-chain CRISPR RNA (crRNA) complementary to a target nucleotide sequence and a trans-activated crRNA (tracrRNA) or a single synthetic RNA (guide RNA, gRNA) obtained by combining the crRNA and the tracrRNA, an arbitrary sequence can be targeted only by synthesizing an oligo DNA capable of specifically hybridizing with the complementary sequence of the target nucleotide sequence.

A nucleic acid sequence-recognizing module using CRISPR-Cas is provided as a complex of an RNA molecule (guide RNA) including a nucleotide sequence complementary to a target nucleotide sequence and tracrRNA necessary for recruitment of a Cas protein and a Cas protein. As another aspect, a nucleic acid sequence-recognizing module using CRISPR-Cas is provided as a complex of crRNA including an RNA sequence complementary to a target nucleotide sequence, tracrRNA, and Cas.

The Cas protein used in the present invention is not particularly limited as long as it belongs to the CRISPR system, but is preferably Cas9. Examples of Cas9 include, but are not limited to, Cas9 (SpCas9) derived from Streptococcus pyogenes and Cas9 (StCas9) derived from Streptococcus thermophilus. SpCas9 is preferred. In consideration of use in human clinic, generation of DSB is not preferable, and thus Cas is preferably one in which the DNA cleavage activity is deactivated (dCas). For example, in the case of SpCas9, a double mutant of a D10A mutant in which the Asp residue at position 10 is converted into an Ala residue and which lacks the ability to cleave the opposite strand of the strand forming the complementary strand with the guide RNA and an H840A mutant in which the His residue at position 840 is converted into an Ala residue and which lacks the ability to cleave the complementary strand with the guide RNA can be used, but other mutation Cas can be used as well.

When CRISPR-Cas is used as a nucleic acid sequence-recognizing module, the target nucleotide sequence is not particularly limited as long as when a complex of a guide RNA and Cas is bound to the sequence, the LSMEM2 gene is blocked by the complex, and the LSMEM2 gene cannot be expressed, and for example, in the case of the LSMEM2 gene, examples thereof include a target nucleotide sequence for a guide RNA (ACTGGCTCCTAACCACGTGCAGG) (SEQ ID NO: 5) used in Examples described later. On the other hand, in the case of the LSMEM2 gene, for example, crRNA can be designed using CGG in the sequence as PAM and a reverse chain sequence of 20 nucleotides on the upstream side immediately before the CGG as a target nucleotide sequence.

In another aspect of the present invention, as the nucleic acid sequence-recognizing module, in addition to a zinc finger motif, a TAL effector, a PPR motif, and the like, a fragment containing a DNA binding domain of a protein capable of specifically binding to DNA, such as a restriction enzyme, a transcription factor, or an RNA polymerase, and having no DNA duplex cleaving ability, and the like can be used.

For details of production of the zinc finger motif, JP 4968498 B2 can be referred to. For details of production of the TAL effector, JP 2013-513389 A can be referred to. For details of production of the PPR motif, JP 2013-128413 A can be referred to.

In the case of using fragments such as a restriction enzyme, a transcription factor, and an RNA polymerase, since the DNA binding domains of these proteins are well known, fragments containing the domains and having no DNA duplex cleaving ability can be easily designed and constructed.

Alternatively, as in the case of dCas of CRISPR-Cas described above, by combining a deactivated nuclease (e.g., FokI) with a nucleic acid sequence-recognizing module such as a zinc finger motif, a TAL effector, or a PPR motif, the nuclease can sterically block the LSMEM2 gene, thereby inhibiting the expression of LSMEM2. In this case, for example, two nucleic acid sequence-recognizing modules that specifically recognize a positive-chain nucleotide sequence upstream of a target region and a reverse-chain nucleotide sequence downstream of the target region can be designed to form a complex with a nuclease deactivated at the end of the module (e.g., C-terminus).

A nucleic acid sequence-recognizing module such as a zinc finger motif, a TAL effector, and a PPR motif can be provided as a fusion protein with the nuclease, or a protein binding domain such as an SH3 domain, a PDZ domain, a GK domain, or a GB domain and a binding partner thereof may be fused to a nucleic acid sequence-recognizing module and a nuclease, respectively, and provided as a protein complex via interaction between the protein binding domain and the binding partner thereof. Alternatively, a nucleic acid sequence-recognizing module and a nuclease can be fused with intein, respectively, and the nucleic acid sequence-recognizing module and the nuclease can be linked to each other by ligation after synthesis of each protein.

The nucleic acid sequence-recognizing module of the present invention can further inhibit transcription of the LSMEM2 gene while blocking binding of the transcription factor to the cis element of the LSMEM2 gene by forming a complex with a transcription repressor instead of a nuclease and using a sequence containing the cis element in the transcription regulatory region of the LSMEM2 gene as a target nucleotide sequence. In the present invention, the “transcription repressor” means a protein or a protein domain having a transcription repression activity of a target gene.

The transcription repressor used in the present invention is not particularly limited as long as it can inhibit transcriptional activation of the LSMEM2 gene, and examples thereof include KRAB, MBD2B, v-ErbA, SID (including concatemer of SID(SID4X)), MBD2, MBD3, DNMT family (e.g.: DNMT1, DNMT3A, DNMT3B), Rb, MeCP2, ROM2, and AtHD2A, and KRAB is preferable. When KRAB is used as a transcription repressor, the protein from which KRAB is derived is not particularly limited, and examples thereof include KOX-1(ZNF10), KOX8(ZNF708), ZNF43, ZNF184, ZNF91, HPF4, HTF10, and HTF34.

The contact between the nucleic acid sequence-recognizing module and the LSMEM2 gene is performed by introducing a nucleic acid encoding the module (when used in combination with an effector such as a nuclease or a transcription repressor, the effector protein) into a cell of a mammalian subject (e.g., human, mouse, rat, cow, dog, cat, monkey, etc., preferably human or mouse, more preferably human).

Therefore, it is preferable to prepare the nucleic acid sequence-recognizing module of the nucleic acid sequence-recognizing module and the effector as a nucleic acid encoding these fusion proteins, or a nucleic acid encoding each constituent in a form capable of forming a complex in a host cell after translation into a protein. Here, the nucleic acid may be DNA or RNA. In the case of DNA, preferably double-stranded DNA is provided in the form of an expression vector capable of expressing each constituent under the control of a functional promoter in a mammalian cell. In the case of RNA, single-stranded RNA is preferable.

When CRISPR-Cas is used as a nucleic acid sequence-recognizing module, an expression vector encoding a guide RNA and a Cas protein is introduced into a cell, and the guide RNA and the Cas protein are expressed to form a complex of the guide RNA and the Cas protein in the cell. The guide RNA and the Cas protein may be encoded on the same expression vector or may be encoded on different expression vectors, respectively.

The DNA encoding Cas can be cloned from cells producing Cas by methods well known in the art. The resulting DNA encoding Cas can be inserted downstream of the promoter of an expression vector for mammalian cells.

On the other hand, the DNA encoding the guide RNA can be chemically synthesized by designing an oligo DNA sequence in which an RNA sequence complementary to the target nucleotide sequence and a known tracrRNA sequence (for example, gttttagagctagaaatagcaagttaaaataaggctagtccgttatcaacttgaaaaagtggca ccgagtcggtggtgctttt; SEQ ID NO: 6) are linked, and using a DNA/RNA synthesizer. In the present specification, the nucleotide sequence is described as a sequence of DNA unless otherwise specified, but when the polynucleotide is RNA, thymine (T) is appropriately replaced with uracil (U).

The DNA encoding the guide RNA can also be inserted into an expression vector for mammalian cells. The guide RNA and the Cas may be encoded on the same expression vector or may be encoded on different expression vectors, respectively. Preferably, the DNA encoding Cas and the DNA encoding the guide RNA are inserted downstream of separate promoters in the same expression vector.

The RNA encoding Cas can also be prepared, for example, by transcribing a DNA encoding Cas described above as a template into mRNA in an in vitro transcription system known per se.

The guide RNA can also be chemically synthesized by designing an oligo RNA sequence in which an RNA sequence complementary to the target nucleotide sequence and a known tracrRNA sequence are linked, and using a DNA/RNA synthesizer. In this case, various modifications for improving stability and membrane permeability can be imparted to the ribonucleotide constituting the guide RNA. Alternatively, crRNA and tracrRNA can also be separately synthesized and annealed for use.

The DNA encoding a nucleic acid sequence-recognizing module such as a zinc finger motif, a TAL effector, or a PPR motif can be obtained by any of the methods described above for each module. The DNA encoding a sequence recognizing module such as a restriction enzyme, a transcription factor, or an RNA polymerase can be cloned, for example, by synthesizing an oligo DNA primer so as to cover a region encoding a desired portion (a portion including a DNA binding domain) of the protein on the basis of cDNA sequence information, and amplifying the DNA by RT-PCR method using total RNA or mRNA fraction prepared from a cell producing the protein as a template.

Similarly, the DNA encoding an effector such as a nuclease or a transcription repressor can be cloned by synthesizing an oligo DNA primer on the basis of cDNA sequence information of the effector to be used, and amplifying the DNA by RT-PCR method using total RNA or mRNA fraction prepared from a cell producing the effector as a template. For example, the DNA encoding FokI can be cloned from mRNA derived from Flavobacterium okeanokoites (IFO 12536) by RT-PCR method by designing appropriate primers for upstream and downstream of CDS based on its cDNA sequence.

The DNA encoding the cloned nucleic acid sequence-recognizing module can be digested directly or optionally with restriction enzymes or added with appropriate linkers and/or nuclear localization signals. In the case of use in combination with an effector such as a nuclease or a transcription repressor, DNA encoding a cloned nucleic acid sequence-recognizing module can be ligated with the DNA encoding the cloned nucleic acid sequence-recognizing module to prepare a DNA encoding a fusion protein. Alternatively, DNA encoding the nucleic acid sequence-recognizing module and DNA encoding the effector may be fused with DNA encoding a binding domain or a binding partner thereof, respectively, or DNA encoding a separation intein may be fused with both DNAs, so that a complex can be formed after the nucleic acid sequence-recognizing module and the effector are translated in a host cell. Also in these cases, a linker and/or a nuclear localization signal can be linked to an appropriate position of one or both DNAs as desired.

The DNA encoding the nucleic acid sequence-recognizing module and the DNA encoding the effector can also construct DNA encoding the entire length by chemically synthesizing DNA strands or connecting synthesized partially overlapping oligo-DNA short strands using a PCR method or a Gibson Assembly method. The advantage of constructing full-length DNA by chemical synthesis or a combination with a PCR method or a Gibson Assembly method is that the use codon can be designed over the entire length of CDS according to the host into which the DNA is introduced. In expression of heterologous DNA, by converting the DNA sequence into a codon that is frequently used in a host organism, an increase in protein expression level can be expected. For data of the codon use frequency in the host to be used, for example, a genetic code use frequency database published on the homepage of Kazusa DNA Research Institute can be used, or a document describing the codon use frequency in each host may be referred to. With reference to the obtained data and the DNA sequence to be introduced, among codons used in the DNA sequence, a codon having a low use frequency in the host may be converted into a codon encoding the same amino acid and having a high use frequency.

Examples of the expression vector into which the DNA encoding a nucleic acid sequence-recognizing module and/or an effector is inserted include animal virus vectors such as retrovirus, vaccinia virus, and adenovirus.

As the promoter, a promoter capable of functioning in mammalian cells, such as an SRα promoter, an SV40 promoter, an LTR promoter, a CMV (cytomegalovirus) promoter, an RSV (Rous sarcoma virus) promoter, a MoMuLV (Moloney murine leukemia virus) LTR, HSV-TK (herpes simplex virus thymidine kinase) promoter, is used, but the promoter is not limited thereto.

In addition to the above, the expression vector may optionally contain an enhancer, a splicing signal, a terminator, a polyA addition signal, a selection marker such as a drug resistance gene, a replication origin, and the like.

By introducing an expression vector encoding a nucleic acid sequence-recognizing module (and optionally further an effector) into a target cell of a target mammal, a nucleic acid sequence-recognizing module or a complex of the module and an effector is expressed and formed in the cell, and can be brought into contact with a target nucleotide sequence on the LSMEM2 gene.

(d-2) Binding Inhibiting Substance with Modification of Genomic Sequence

In the above (d), by using a wild-type enzyme having DSB activity as a nuclease, DSB is generated in the LSMEM2 gene, and deletion of one or more nucleotides of the LSMEM2 gene, substitution with another nucleotide, or insertion of one or more nucleotides into the gene is caused, whereby the LSMEM2 gene can be disrupted.

The LSMEM2 gene is cleaved within the target nucleotide sequence and then repaired by non-homologous end joining (NHEJ), but due to the repair error at this time, deletion of one or more nucleotides of the gene, substitution with another nucleotide, or insertion of one or more nucleotides into the gene is caused. As a result, a mutation (e.g., frameshift mutation) can be generated in the LSMEM2 gene, and the expression of correctly translated LSMEM2 mRNA can be inhibited.

As another embodiment in which a mutation is introduced into the LSMEM2 gene, a DNA (donor DNA) including sequences homologous to the upstream and downstream sequences adjacent to the target region in the LSMEM2 gene and having a mutation introduced into the target region portion is introduced into a mammalian cell of interest together with a DNA encoding a complex of a nucleic acid sequence-recognizing module and a nuclease, whereby a desired mutation can be introduced into the target region by causing homologous recombination between the region including the target region of the present invention and the donor DNA. The sequences homologous to the upstream and downstream sequences adjacent to the target region included in the donor DNA are not particularly limited as long as they have a length sufficient to cause homologous recombination, and may be a relatively short sequence of about 50 to 100 mers or a long homology arm of several kb. In the latter case, the donor DNA may be provided in the form of a targeting vector into which the donor DNA has been inserted. The “targeting vector into which the donor DNA has been inserted” is not limited to simply a targeting vector into which the same sequence as that of the donor DNA is inserted, and includes those having a selective marker and/or a recombinase target sequence between or outside the donor DNA. The vector serving as the basic skeleton of the targeting vector is not particularly limited, and any vector may be used as long as it can self-replicate in a cell to be transformed (for example, E. coli). For example, commercially available pBluscript (manufactured by Stratagene), pZErO1.1 (Invitrogen), pGEM-1 (Promega), and the like can be used.

The donor DNA can be introduced into the cell in any form of double-stranded DNA (cyclic double-stranded DNA or linear double-stranded DNA) or single-stranded DNA.

Since DSB involves unexpected genome modification, there are side effects such as strong cytotoxicity and chromosomal translocation, and reliability in gene therapy may be impaired. Therefore, instead of the nuclease, an enzyme capable of genome modification without DSB as an effector can be used in combination with the nucleic acid sequence-recognizing module. Examples of such an enzyme include, but are not limited to, an enzyme that converts a substituent of a nucleic acid base to another substituent to substitute a different base (e.g., deaminase, etc.), and an enzyme that catalyzes an abasic reaction and introduces a mutation at an abasic site using an error in an intrinsic repair mechanism (e.g., DNA glycosylase, etc.). In this case, when the nucleic acid sequence-recognizing module is CRISPR-Cas, a mutant (nCas) in which at least one DNA cleaving ability is deactivated, preferably a mutant (dCas) in which both DNA cleaving abilities are deactivated, is used as Cas. Details in the case of using a deaminase as an effector are described, for example, in WO 2015/133554 A, and details in the case of using a DNA glycosylase are described, for example, in WO 2016/072399 A.

Without being bound by theory, the myocardium under an extension load expresses inflammatory cytokines in a mechanostress-dependent manner, and induces inflammation in the myocardium. This becomes a vicious cycle and further deteriorates the cardiac function and deteriorates the prognosis, but it is considered that LSMEM2 inhibition exhibits a therapeutic effect by breaking this vicious cycle. It has been found that LSMEM2 interacts with VPS35, which is a constituent of a retromer complex that plays a central role in vesicle transport, in laminopathies. VPS35 is a central molecule in vesicle transport that controls the orientation of vesicles by interacting molecules. On the other hand, it has been reported that SUN1, which is a nuclear membrane protein related to mechanotransduction, is mislocalized in the Golgi body in the laminopathy model cells (Cell. 2012; 149(3): 565-77), and it is strongly suggested that an abnormality in vesicle transport is involved in the pathogenesis of laminopathies. Since LSMEM2 deficiency improves the phenotype of laminopathies, it is considered that the vesicle transport modification involved in the pathogenesis of laminopathies and the LSMEM2-dependent vesicle transport modification in the presence of mechanostress overlap. The “substance inhibiting the function of LSMEM2” in the present invention may be any substance as long as it inhibits the function contributing to mechanosignaling of LSMEM2 once functionally produced, and examples thereof include a substance binding to LSMEM2 and inhibiting the function, and a substance inhibiting the migration of LSMEM2 to a cell membrane.

Specific examples of the substance inhibiting the function of LSMEM2 include an antibody against LSMEM2. The antibody may be either a polyclonal antibody or a monoclonal antibody. These antibodies can be produced according to a method for producing an antibody or antiserum known per se. The isotype of the antibody is not particularly limited, but IgG, IgM, or IgA is preferable, and IgG is particularly preferable. The antibody is not particularly limited as long as it has at least a complementarity determining region (CDR) for specifically recognizing and binding to LSMEM2, and may be, for example, a fragment such as Fab, Fab′, or F(ab′)₂, a conjugate molecule produced by genetic engineering such as scFv, scFv-Fc, a minibody, or a diabody, a derivative thereof modified with a molecule having a protein stabilizing action such as polyethylene glycol (PEG), or the like, in addition to a complete antibody molecule.

In a preferred embodiment, since the antibody against LSMEM2 is used as a pharmaceutical product to be administered to a human, the antibody (preferably, a monoclonal antibody) is an antibody having a reduced risk of exhibiting antigenicity in the case of being administered to a human, specifically, a fully human antibody, a humanized antibody, a mouse-human chimeric antibody, or the like, and particularly preferably a fully human antibody. The humanized antibody and the chimeric antibody can be produced by genetic engineering according to a conventional method. The fully human antibody can be produced from a human-human (or mouse) hybridoma, but in order to stably provide a large amount of antibody at low cost, it is desirable to produce the fully human antibody using a human antibody-producing mouse or a phage display method.

The substance inhibiting the function of LSMEM2 may also be, for example, an aptamer (peptide aptamer or nucleic acid aptamer). An aptamer can be screened and produced according to a well-known method (for example, Ellington et al., (1990), Nature, 346, 818-822; Tuerk et al., (1990) Science, 249, 505-510).

The substance inhibiting the function of LSMEM2 may also be, for example, a substance inducing decomposition of LSMEM2. Examples of the substance inducing decomposition of LSMEM2 include LSMEM2 degraders using a targeted protein degradation technique. Such a targeted protein degrader can be produced according to a well-known method (for example, Mohammadsharif Tabebordbar et al., Cell. 2021 Sep. 16; 184(19): 4919-4938. e22.).

In the present specification, the “mechanosignaling” means that when a cell receives a mechanical stimulus (e.g. fluid pressure, extension stimulation, or the like), a structural change is caused in a sensor molecule (PIEZO) that is a component of the cell, whereby information of the mechanical stimulus is converted into a biochemical signal, and transmitted by other mediated signal molecules so as to cause sensing, interpretation, response, and the like by the cell.

As shown in Examples described later, since LSMEM2 is necessary and sufficient for mechano-signal (mechanostress (extension stimulation)-dependent inflammatory signal) transmission, the substance inhibiting the expression or function of LSMEM2 has an action of inhibiting mechanosignaling, and can be used as a prophylactic and/or therapeutic agent for dilated cardiomyopathy as a target therapeutic agent for cardiomyocytes and the like. Since a mouse generated by crossbreeding a LSMEM2 knockout mouse with a cardiolaminopathy model mouse has improved cardiac function and lower limb muscle strength, a substance inhibiting the expression or function of LSMEM2 can also be used as a prophylactic and/or therapeutic agent for laminopathies. Since LSMEM2 KO mice develop normally, have completely normal cardiac function, and no problems with reproductive function, the substance inhibiting the expression or function of LSMEM2 has an additional advantageous effect of being less toxic to normal cells and having a low risk of side effects. Therefore, a medicament containing a substance inhibiting the expression or function of LSMEM2 is useful as a mechanosignaling inhibitor, and further, as a prophylactic and/or therapeutic agent for heart diseases, and is also useful as a prophylactic and/or therapeutic agent for laminopathies. Here, the term “treatment” is used in the meaning of including improvement in disease and improvement in prognosis. The term “prevention” is used in the meaning of including delay of onset of a disease.

The substance inhibiting the expression or function of LSMEM2 may be used singly, or may be used in combination of two or more thereof. Two or more substances inhibiting the expression or function of LSMEM2 may be formulated as separate medicaments, or may be formulated in the same pharmaceutical composition. When two or more substances inhibiting the expression or function of LSMEM2 are formulated as separate medicaments, each preparation may be administered simultaneously or may be administered after a lapse of time. The administration routes may be the same or different. The dose described later indicates the dose of one substance inhibiting the expression or function of LSMEM2, but even when two or more substances are used in combination, the same dose can be used for each substance as long as it does not adversely affect the administration subject.

(1) Medicament Containing siRNA and Precursor, Antisense Nucleic Acid, and Ribozyme Nucleic Acid Thereof

An antisense nucleic acid (or miRNA) that can complementarily bind to a transcription product of an LSMEM2 gene to inhibit translation of a protein from the transcription product, siRNA (or ribozyme, miRNA) that can cleave a transcription product of the LSMEM2 gene targeting a homologous (or complementary) base sequence in the transcription product, shRNA or pre-miRNA, which is a precursor of the siRNA or miRNA, and the like (hereinafter, comprehensively referred to as “nucleic acid of the present invention” in some cases) can be used as an anti-inflammatory agent and/or an inhibitor of abnormal vesicle trafficking, and further, as a prophylactic and/or therapeutic agent for heart diseases and/or a prophylactic and/or therapeutic agent for laminopathies.

The medicament containing the nucleic acid of the present invention can be administered to a subject such as a human or a non-human warm-blooded animal (e.g., rat, rabbit, sheep, pig, cow, cat, dog, monkey, chicken, etc.) orally or parenterally (e.g., intravascular administration, subcutaneous administration, etc.) as a liquid preparation as it is or as a pharmaceutical composition of an appropriate dosage form.

When the nucleic acid of the present invention is used as an anti-inflammatory agent and/or an inhibitor of abnormal vesicle trafficking, and further, as a prophylactic and/or therapeutic agent for heart diseases and/or a prophylactic and/or therapeutic agent for laminopathies, the nucleic acid can be formulated and then administered according to a method known per se. That is, the nucleic acid of the present invention may be used singly, or can also be inserted into an appropriate expression vector for mammalian cells, such as a retroviral vector, an adenoviral vector, or an adenoviral associated viral vector, in an operable manner. The nucleic acid can be administered as it is or together with an auxiliary agent for promoting intake by a catheter such as a gene gun or a hydrogel catheter. Alternatively, it can be aerosolized and topically administered into the trachea as an inhalant.

For the purpose of improving the pharmacokinetics, prolonging the half-life, and improving the intracellular uptake efficiency, the nucleic acid may be formulated (injected) singly or together with a carrier such as liposome, and administered intravenously, subcutaneously, or the like.

The nucleic acid of the present invention may be administered per se or as a suitable pharmaceutical composition. The pharmaceutical composition used for administration may contain the nucleic acid of the present invention and a pharmacologically acceptable carrier, diluent, or excipient. Such a pharmaceutical composition is provided as dosage forms suitable for oral or parenteral administration.

As the composition for parenteral administration, for example, an injection, a suppository, an intranasal administration agent, and the like are used, and the injection may include dosage forms such as an intravenous injection, a subcutaneous injection, an intradermal injection, an intramuscular injection, and a drip injection. Such an injection can be prepared according to a known method. As a method for preparing an injection, for example, the nucleic acid of the present invention can be prepared by dissolving, suspending or emulsifying the nucleic acid of the present invention in a sterile aqueous solution or oily solution usually used for an injection. As the aqueous solution for injection, for example, an isotonic solution containing physiological saline, glucose, or other adjuvant is used, and may be used in combination with an appropriate solubilizing agent, for example, alcohol (e.g., ethanol), polyalcohol (e.g., propylene glycol or polyethylene glycol), nonionic surfactant [e.g., Polysorbate 80, HCO-50 (polyoxyethylene (50 mol) adduct of hydrogenated castor oil)], or the like. As the oily solution, for example, sesame oil, soybean oil, or the like is used, and benzyl benzoate, benzyl alcohol, or the like may be used in combination as a solubilizing agent. The prepared injection solution is preferably filled in an appropriate ampoule. The suppository used for rectal administration may be prepared by mixing the nucleic acid with a normal suppository base.

Examples of the composition for oral administration include solid or liquid dosage forms, specifically, tablets (including dragee tablets and film-coated tablets), pills, granules, powders, capsules (including soft capsules), syrups, emulsions, and suspensions. Such a composition is produced by a known method, and may contain a carrier, a diluent, or an excipient usually used in the field of formulation. Examples of the carrier and excipient for tablets include lactose, starch, sucrose, and magnesium stearate.

It is advantageous that the parenteral or oral pharmaceutical composition is prepared in a dosage form of a dosage unit to suit the dosage of the active ingredient. Examples of the dosage form of such a dosage unit include tablets, pills, capsules, injections (ampoules), and suppositories. The nucleic acid of the present invention is preferably contained, for example, usually in an amount of about 0.01 to 500 mg per unit dosage form.

The dose of the medicament containing the nucleic acid of the present invention varies depending on the administration subject, the target disease, the symptom, the administration route, and the like, and for example, when the medicament is used in the treatment/prevention of heart diseases or laminopathies, it is convenient to administer the nucleic acid of the present invention as a single dose by intravenous injection usually about 0.0001 to 20 mg/kg body weight once a day to 6 months. Similar amounts can be administered for other parenteral and oral administrations. When the symptoms are particularly severe, the amount may be increased according to the symptoms.

### (2) Medicament Containing Nucleic Acid Sequence-Recognizing Module

When the binding inhibitor of the present invention is a nucleic acid sequence-recognizing module (and an effector forming a complex with the module) utilized in genome editing technique, preferably, the binding inhibiting substance is formulated in the form of an expression vector (hereinafter, also referred to as “vector of the present invention”) containing a DNA encoding the binding inhibiting substance. As the vector of the present invention, for example, a viral vector such as detoxified retrovirus, adenovirus, adeno-associated virus, herpes virus, vaccinia virus, poxvirus, poliovirus, Sindbis virus, Sendai virus, SV40, or immunodeficiency virus (HIV) can be used. Preferably, adenovirus or adeno-associated virus vectors are mentioned.

Examples of the pharmacologically acceptable carrier include, but are not limited to, excipients such as sucrose and starch, binders such as cellulose and methyl cellulose, disintegrants such as starch and carboxymethyl cellulose, lubricants such as magnesium stearate and aerodyl, fragrances such as citric acid and menthol, preservatives such as sodium benzoate and sodium hydrogen sulfite, stabilizers such as citric acid and sodium citrate, suspending agents such as methyl cellulose and polyvinyl pyrrolide, dispersing agents such as surfactants, diluents such as water and physiological saline, and base waxes.

In order to promote the introduction of the vector into the target cell, the agent of the present invention can further contain a reagent for nucleic acid introduction. As the reagent for nucleic acid introduction, atelocollagen; liposome; nanoparticles; cationic lipids such as lipofectin, lipofectamine, DOGS (transfectum), DOPE, DOTAP, DDAB, DHDEAB, HDEAB, polybrene, or poly(ethyleneimine) (PEI), and the like can be used.

In a preferred embodiment, the agent of the present invention may be a pharmaceutical composition in which the vector is encapsulated in a liposome. Liposomes are microclosed vesicles having an internal phase surrounded by one or more lipid bilayers, and can usually retain water-soluble substances in the internal phase and fat-soluble substances in the lipid bilayer. When the term “encapsulation” is used in the present specification, the vector may be retained in the liposome inner phase or may be retained in the lipid bilayer. The liposome used in the present invention may be a monolayer membrane or a multilayer membrane, and the particle size can be appropriately selected in a range of, for example, 10 to 1000 nm, preferably 50 to 300 nm. In consideration of the delivery property to the target tissue, the particle size may be, for example, 200 nm or less, preferably 100 nm or less.

Examples of the method for encapsulating a vector in a liposome include a lipid film method (vortex method), a reverse phase evaporation method, a surfactant removal method, a freeze-thaw method, and a remote loading method, but are not limited thereto, and any known method can be appropriately selected.

The agent of the present invention can be administered to a mammal (e.g., human, rat, mouse, guinea pig, rabbit, sheep, horse, pig, cow, or monkey) orally or parenterally, but is desirably administered parenterally.

It is also possible to prepare a sustained-release preparation (mini pellet formulations or the like) to be embedded close to the diseased part, or the preparation is continuously and gradually administered to the diseased site using an osmotic pump or the like.

As preparations suitable for parenteral administration (for example, subcutaneous injection, intramuscular injection, local injection, intraperitoneal administration, etc.), aqueous and non-aqueous isotonic sterile injectable liquids are available, which may include an antioxidant, a buffer solution, a bacteriostatic agent, an isotonizing agent, and the like. Aqueous and non-aqueous sterile suspensions are also mentioned, which may include a suspending agent, a solubilizer, a thickener, a stabilizer, an antiseptic, and the like. The preparation can be enclosed in a container such as an ampule or a vial in a unit dosage volume or in several divided doses. An active ingredient and a pharmaceutically acceptable carrier can also be freeze-dried and stored in a state that may be dissolved or suspended in an appropriate sterile vehicle just before use.

The content of the vector in the pharmaceutical composition is, for example, about 0.1 to 100 wt % of the total pharmaceutical composition.

The dose of the agent of the present invention varies depending on the administration method, the type and severity of the disease to be administered, and the situation of the administration subject (sex, age, weight, etc.), but for example, when the agent is systemically administered to an adult, in general, when the vector is administered as a viral vector particle, the agent can be administered in a range of, for example, about 1×10³ pfu to 1×10¹⁵ pfu as a viral titer per administration.

When the substance is provided in the form of an expression vector containing a DNA encoding the substance, the agent of the present invention may be an ex vivo preparation in which the substance is added to a cell or tissue (e.g., cardiomyocytes) collected from an administration subject, the expression vector is introduced into the cell, and the expression vector is returned to the body of the administration subject, preferably to a heart lesion. In this case, examples of the method for introducing a gene into a cell include a lipofection method, a phosphate-calcium coprecipitation method; and a direct injection method using a microglass tube. Examples of the method for introducing a gene into a tissue include a gene introduction method using an encapsulated liposome, a gene introduction method using an electrostatic liposome, an HVJ-liposome method, a improved HVJ-liposome method (HVJ-AVE liposome method), a receptor-mediated gene introduction method, a method in which an active ingredient is transferred into a cell together with a carrier (metal particle) using a particle gun, a direct introduction method of naked-DNA, and an introduction method using a positively charged polymer.

### (3) Medicament Containing Antibody Against LSMEM2, Low-Molecular-Weight Compound Inhibiting Expression or Function of LSMEM2, or LSMEM2 Degrader

The antibody against LSMEM2, the LSMEM2 degrader, or the low-molecular-weight compound inhibiting the expression or function of LSMEM2 can inhibit the function contributing to the production of LSMEM2 or mechanosignaling thereof. Therefore, these substances can be used as a mechanosignaling inhibitor, and further, as a prophylactic and/or therapeutic agent for heart diseases and/or a prophylactic and/or therapeutic agent for laminopathies.

The medicament containing the antibody, the degrader, or the low-molecular-weight compound can be administered to a human or other warm-blooded animals (e.g., rat, rabbit, sheep, pig, cow, cat, dog, monkey, chicken, etc.) orally or parenterally (e.g., intravascular administration, subcutaneous administration, or the like) as a liquid preparation as it is or as a pharmaceutical composition of an appropriate dosage form.

The antibody, degrader, or low-molecular-weight compound may be administered per se or as a suitable pharmaceutical composition. The pharmaceutical composition used for administration may contain the antibody or low-molecular-weight compound or a salt thereof and a pharmacologically acceptable carrier, diluent, or excipient. Such a pharmaceutical composition is provided as dosage forms suitable for oral or parenteral administration.

As the composition for parenteral administration, for example, an injection, a suppository, an intranasal administration agent, and the like are used, and the injection may include dosage forms such as an intravenous injection, a subcutaneous injection, an intradermal injection, an intramuscular injection, and a drip injection. Such an injection can be prepared according to a known method. As a method for preparing an injection, for example, the nucleic acid of the present invention can be prepared by dissolving, suspending or emulsifying the antibody of the low-molecular-weight compound of the present invention or a salt thereof in a sterile aqueous solution or oily solution usually used for an injection. As the aqueous solution for injection, for example, an isotonic solution containing physiological saline, glucose, or other adjuvant is used, and may be used in combination with an appropriate solubilizing agent, for example, alcohol (e.g., ethanol), polyalcohol (e.g., propylene glycol or polyethylene glycol), nonionic surfactant [e.g., Polysorbate 80, HCO-50 (polyoxyethylene (50 mol) adduct of hydrogenated castor oil)], or the like. As the oily solution, for example, sesame oil, soybean oil, or the like is used, and benzyl benzoate, benzyl alcohol, or the like may be used in combination as a solubilizing agent. The prepared injection solution is preferably filled in an appropriate ampoule. The suppository used for rectal administration may be prepared by mixing the antibody or a salt thereof with a normal suppository base.

Examples of the composition for oral administration include solid or liquid dosage forms, specifically, tablets (including dragee tablets and film-coated tablets), pills, granules, powders, capsules (including soft capsules), syrups, emulsions, and suspensions. Such a composition is produced by a known method, and may contain a carrier, a diluent, or an excipient usually used in the field of formulation. Examples of the carrier and excipient for tablets include lactose, starch, sucrose, and magnesium stearate.

It is advantageous that the parenteral or oral pharmaceutical composition is prepared in a dosage form of a dosage unit to suit the dosage of the active ingredient. Examples of the dosage form of such a dosage unit include tablets, pills, capsules, injections (ampoules), and suppositories. It is preferable that the antibody and the low-molecular-weight compound are contained in an amount of usually 0.1 to 500 mg per unit dosage form, particularly 5 to 100 mg for injections, and 10 to 250 mg for other dosage forms.

The dose of the medicament containing the antibody or the low-molecular-weight compound or a salt thereof varies depending on the administration subject, the target disease, the symptom, the administration route, and the like, and for example, it is convenient to administer the antibody or the low-molecular-weight compound by intravenous injection usually at about 0.0001 to 20 mg/kg body weight as a single dose about 1 to 5 times a day for the low-molecular-weight compound, orally or parenterally, or once a day to several months for the antibody. Similar amounts can be administered for other parenteral and oral administrations. When the symptoms are particularly severe, the amount may be increased according to the symptoms.

Each of the compositions described above may contain other active ingredients as long as an undesirable interaction is not caused by blending with the antibody or the low-molecular-weight compound. Examples thereof include antibacterial agents, antifungal agents, non-steroidal antiinflammatory drugs, steroids, anticoagulants, platelet aggregation inhibitors, thrombolytic drugs, immunomodulators, antiprotozoals, antibiotics, antiviral drugs, antitussive and expectorant drugs, sedatives, anesthetics, antiulcer drugs, antiarrhythmics, hypotensive diuretics, tranquilizers, antipsychotics, antitumor drugs, hypolipidemic drugs, muscle relaxants, anticonvulsants, antidepressants, antiallergic drugs, cardiacs, antiarrhythmics, vasodilators, vasoconstrictors, hypotensive diuretics, antidiabetic drugs, antinarcotics, vitamins, vitamin derivatives, therapeutic agents for arthritis, antirheumatics, antiasthmatics, therapeutic agents for pollakisuria/anischuria, therapeutic agents for atopic dermatitis, therapeutic agents for allergic rhinitis, hypertensive drugs, protease drugs, protease inhibitors, anti-SIDS drugs, anti-sepsis drugs, anti-septic shock drugs, endotoxin-antagonists or -antibodies, signal transduction inhibitors, inhibitors of inflammatory mediator activity, antibodies to inhibit inflammatory mediator activity, inhibitors of inflammatory mediator production, inhibitors of anti-inflammatory mediator activity, antibodies to inhibit anti-inflammatory mediator activity, inhibitors of anti-inflammatory mediator production, and al-adrenergic agonists. The antibody or the low-molecular-weight compound, and other drugs may be administered to a patient at the same time or at different times.

The medicament can be used, for example, as an anti-inflammatory agent or an inhibitor of abnormal vesicle trafficking, as a prophylactic and/or therapeutic agent for various inflammatory diseases (for example, Crohn's disease, rheumatoid arthritis, Behcet's disease (ocular condition), ulcerative colitis, ankylosing spondylitis, psoriasis (including psoriatic arthritis), HIV infection, multiple myeloma, heart disease (for example, congestive heart failure, chronic heart failure, dilated cardiomyopathy, hypertrophic cardiomyopathy, myocardial ischemia, myocardial infarction, angina pectoris), GVHD, giant cell arteritis (GCA), polymyalgia rheumatica (PMR), pigmented lichenoid dermatitis, sarcoidosis, Wegener's granuloma, pyoderma, Behcet's disease, TNF receptor-associated periodic syndrome (TRAPS), SAPHO syndrome, Takayasu's disease, myositis, Still's disease, Periarteritis nodosa (PN), recurrent polychondritis, scleroderma polymyositis, hemophagocytic syndrome, pemphigus, Kawasaki atopic dermatitis), and laminopathies (for example, muscular dystrophy, cardiolaminopathy, lipodystrophy, leukodystrophy), and the like.

Hereinafter, the present invention will be described in detail with reference to Examples and the like, but the present invention is not limited thereto.

### EXAMPLE

### Example 1

### Mouse Tissue RT-PCR

RT-PCR was performed to see the expression kinetics of LSMEM2 in mouse and human tissues. As cDNA, Mouse MTC Panel I (636745) from Clontech Laboratories was used, and heart, brain, liver, skeletal muscle, testis, kidney, spleen, and lung were examined. TaKaRa Ex Taq (Takara Bio Inc. Shiga, Japan) was used for PCR. Primers and reaction solutions were prepared as follows.

### Primers Used

Mouse_RT-PCR_L agcttctgggtgtcttaccaccatc (SEQ ID NO: 7)
Mouse_RT-PCR_R caatttgagcagagtctcctcttgtgtgc (SEQ ID NO: 8)

TABLE 1

| | |
|---|---|
| Nuclease free water | 5 μL |
| Ex Taq buffer | 2 μL |
| dNTPs | 1.6 μL |
| Primer R | 1 μL |
| Primer L | 1 μL |
| Betain | 4 μL |
| Ex Taq | 0.4 μL |
| cDNA (x10 diluted) | 5 μL |
| | 20 μL |

PCR conditions were performed as follows.

TABLE 2

| | | |
|---|---|---|
| 1 | 94 °C | 120 sec |
| 2 | 94 °C | 30 sec |
| 3 | 66 °C | 40 sec |
| 4 | 72 °C | 120 sec |
| 5 | 72 °C | 300 sec |
| 6 | 4 °C | |
| | | repeat 33 cycles of 2 ⁻4 |

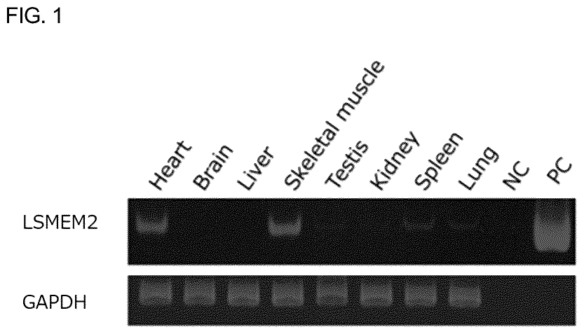

As a result, it was shown that LSMEM2 expression was localized in the myocardium and skeletal muscle (FIG. 1).

### Example 2

### RNA-Seq of LSMEM2 Overexpressing Cardiomyocytes

### Primary Culture of Neonatal Rat Cardiomyocytes

Hearts were extracted from neonatal rats and loosened in Hank's balanced salt solution (Thermo Fisher Scientific, Waltham, MA), and internal blood was washed. The loosened hearts were incubated overnight at 4 °C in 0.25% Trypsin/EDTA (SIGMA, St. Louis, MO). The next day, D-MEM (in 10% FBS) was added and incubated at 37 °C for 5 minutes. The supernatant was discarded, and collagenase was added and mixed by hand for 1 minute. The supernatant was discarded and transferred to a flask, collagenase (Worthington, type 2) was added thereto, and the mixture was stirred for 15 minutes using a stirrer while maintaining the temperature at 37 °C. After centrifugation for 5 minutes at 3000 g, the supernatant was discarded, 30 ml of D-MEM was then added to suspend the cells, and the cells were seeded on a P10 dish and incubated at 37 °C for 70 minutes (in this process, cardiac fibroblasts adhere to the dish, and cardiomyocytes are separated into the culture supernatant). The dish surface was washed with the supernatant to recover cardiomyocytes. Subsequently, the mixture was centrifuged for 5 minutes at 3000 g, the supernatant was discarded, the mixture was suspended in an amount of D-MEM as necessary, a part of the suspension was taken, and the number of cells was counted. Finally, the cells were seeded at an appropriate cell concentration according to the experimental conditions.

### RNA-Seq Analysis of Cardiomyocytes

The cultured cardiomyocytes were prepared to 5.0×10⁶ cell/well and seeded on a 6 cm dish, and after 24 hours, the medium was exchanged, and adenovirus vectors of Wild type LSMEM2, S37A LSMEM2, and Mock were added. After 48 hours, the medium was replaced with serum free D-MEM, and after 16 hours, myocardial RNA was recovered and purified using RNA bee. Subsequently, the RNA-seq data production itself was performed by requesting the Research Institute for Microbial Diseases of Osaka University, but as an outline, the RNA-seq data production was performed as follows. First, as for RNA for each group, RNA library was prepared by TruSeq Stranded mRNA Library Prep Kit (Illumina, San Diego CA). In practice, mRNA was polyA purified by dT beads, ribosomal RNA was removed by Ribo-zero beads, and then an adaptor sequence was added in one direction. Subsequently, HiSeq 2500 of Illumina sequenced an average of 15 million reads with 75 bp single reads to produce RNA seq data. Subsequently, the quality of the obtained read was confirmed by FASTQC. Next, mapping was performed on Rnor6 of NCBI which is reference genome by Tophat (Kim et al., 2013). The sequence of each sample mapped was assembled by using Cufflinks (Baren et al., 2010), and Cuffdiff (Trapnell et al., 2013) was used to detect an expression-variant gene (FPRK) using a file created from Cufflinks. Enrichment analysis was performed using DAVID (Huang, Sherman, & Lempicki, 2009b) (Huang, Sherman, & Lempicki, 2009a) in order to evaluate what function the list of gene groups created and extracted by Cuffdiff interacts with.

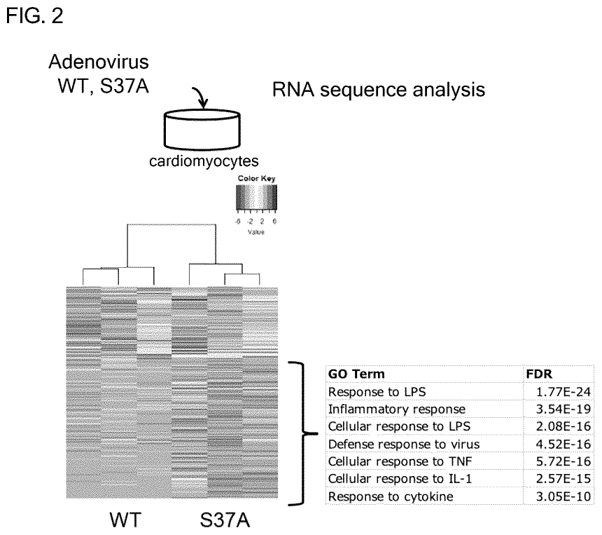

As a result, it was shown that when LSMEM2 was overexpressed in cardiomyocytes, inflammation-related gene expression was increased (FIG. 2).

### Example 3

Quantification of mRNA by Droplet Digital PCR (ddPCR) Method

ddPCR was performed using reagents and apparatuses from Bio-Rad (Bio-Rad Laboratories, Hercules, CA). That is, the prepared sample and Droplet generator oil were mixed in QX200 Droplet Generator to produce Droplet. The produced Droplet was subjected to PCR with a thermal cycler, and a signal was detected with QX200 Droplet Reader. The following probes were used for detection of expression of Rat IL6 and Rat Tbp and detection of expression of Human IL6 and Human Tbp. 116: dRnoCPE5187348(FAM), Tbp: dRnoCNS494181347 (HEX) Human IL6: dHsaCPE5036918, Human TBP: dHsaCPE5058363

TABLE 3

| | |
|---|---|
| Nuclease free water | 7 μL |
| ddPCR ™ Supermix for Probes (No dUTP) | 10 μL |
| ddPCR probe (FAM: 900 nM primers and | 1.0 μL |
| 250 nM probe) | |
| ddPCR probe (HEX: 900 nM primers and | 1.0 μL |
| 250 nM probe) | |
| cDNA (12 ng/μL) | 1.0 μL |
| | 20 μL |

PCR conditions were performed as follows.

TABLE 4

| | | |
|---|---|---|
| 1 | 95°C | 10 min |
| 2 | 94°C | 30 sec |
| 3 | 53°C | 1 min |
| 4 | 98°C | 10 min |
| 5 | 4°C | |
| | | repeat 40 cycles of 2 ⁻3 |

### Cardiomyocyte Hydrostatic Loading

The cultured cardiomyocytes were seeded on a plate, and after 24 hours, the medium replaced with 1% FBS DMEM, 25-mM HEPES-NaOH, pH 7.4 (Sigma-Aldrich, St. Louis, MO), 2-mM 1-glutamine (Thermo Fisher Scientific, Waltham, MA), 1% P/S. The cardiomyocytes were placed in a pressure vessel and connected to a compressor supplying pressurized air adjusted to 37 °C and 5% CO₂. The air compression controller for regulating the flow rate of the air was exposed for 12 hours under hydrostatic conditions where the frequency could be varied from 0 to 0.25 Hz and the amplitude from 0 to 1 MPa, and this time, 101 to 135 kPa was repeated at a cycle of 0.01 Hz. The pressure was applied to the cells by compressing the volume of the medium.

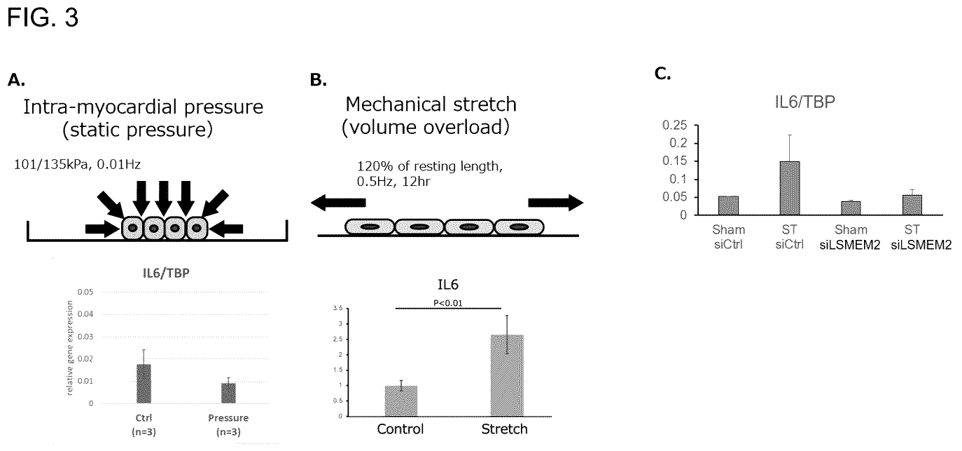

Quantification of the expression of IL6mRNA in the hydrostatically pressure-loaded cardiomyocytes showed that the expression of IL6 was not induced (FIG. 3A; expressed as relative expression to Tbp mRNA).

### Cardiomyocyte and Hela Cell Extension Stimulation Load

A stretch chamber STB-CH-04 (STREX, Osaka, Japan) was subjected to a plasma treatment by a tabletop vacuum plasma treatment apparatus PC-400T (STREX, Osaka, Japan), and then 1 mL of a laminin solution prepared so that iMatrix-511 (Nippi, Tokyo, Japan) was 4.8 μg/mL, was added thereto and incubated at 37 °C overnight. The laminin solution was removed, and cultured cardiomyocytes prepared by adjusting the cultured cardiomyocytes to 1.3×10⁶ cell/chamber were seeded on the stretch chamber. After 24 hours from seeding, the medium was replaced with serum free DMEM, and set in an automatic extension device STB-1400 (STREX, Osaka, Japan), and extension stimulation was applied at 0.5 Hz and 20% for 24 hours under the conditions of a temperature of 37 °C and 5% carbon dioxide.

Quantification of IL6mRNA in the cardiomyocytes loaded with extension stimulation showed that expression of IL6 was induced (FIG. 3B).

LSMEM2 Knockdown in Neonatal Rat Cardiomyocytes Using siRNA

As siRNA for LSMEM2, Silence (registered trademark) rSelect siRNA (LSMEM2 siRNA ID: s189327, sense (5′,3′): GCACAGUGGAGGCUCACUATT (SEQ ID NO: 9) and antisense (5′-3′): UAGUGAGCCUCCACUGUGCAG (SEQ ID NO: 10) (Thermo Fisher Scientific, Waltham, MA) were used. The final concentration was adjusted to 5 nM, and the siRNA was introduced into neonatal rat cardiomyocytes 3 hours after seeding using Lipofectaminem RNAiMAX Transfection Reagent (Thermo Fisher Scientific, Waltham, MA).

IL6 mRNA was quantified in rat cardiomyocytes (siAIPID) in which control siRNA was introduced and rat cardiomyocytes (siCtrl) in which LSMEM2 was knocked down by siRNA, to which extension stimulation was applied. No IL6 expression was induced with control siRNA, but was shown to be induced with siRNA against LSMEM2 (expressed as relative expression against Tbp mRNA; FIG. 3C).

### Example 4

### Production of Lsmem2 Knockout Mice

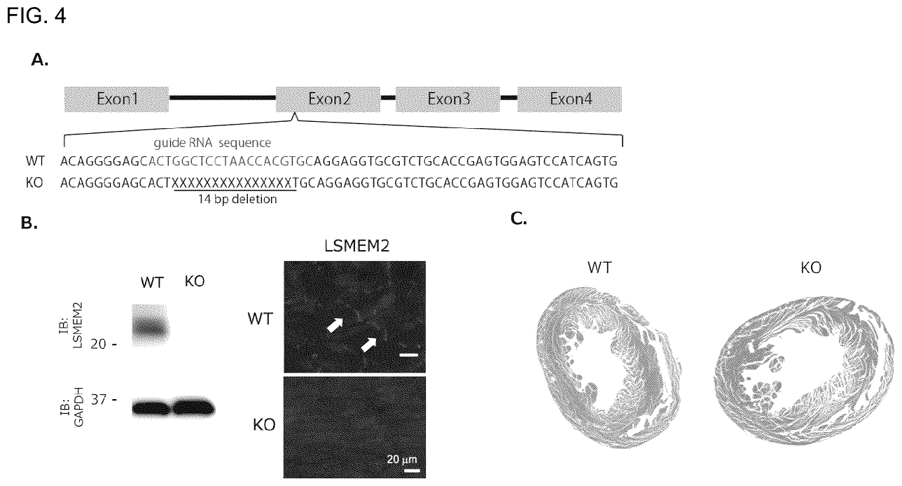

Production of knockout mice was requested to the Reproductive Engineering Unit, Institutes for Animal Experimentation, Osaka University. In practice, LSMEM2 knockout mice were produced using the CRSPR/Cas9 system. Guide RNA was designed on exon 2 (Guide RNA sequence: ACTGGCTCCTAACCACGTGCAGG) (SEQ ID NO: 5) (FIG. 4A). A vector in which the designed Guide RNA was incorporated into pX330 (Wyman et al., 2013) capable of simultaneously expressing Cas9 and target Guide RNA was prepared and injected into a fertilized egg of a mouse, and the fertilized egg was transplanted into a pseudopregnant mouse. The obtained FQ mouse was checked for in-del of the LSMEM2 gene region by Sanger sequence, and a mouse having a deletion of 14 bases was lined up, and subcultured and maintained in a C57BL6J background. LSMEM2 was detected by normal Western blotting using mouse heart tissue, and it was confirmed that LSMEM2 protein disappeared in the knockout mice, that is, the LSMEM2 knockout mice were formed (FIG. 4B).

### Production of Frozen Section of Mouse Heart Tissue

The mouse hearts were excised, washed with PBS, then embedded with O. C. T Compound, and allowed to stand for 5 minutes, and then the hearts were frozen in isopentane cooled with liquid nitrogen, and thereafter, the hearts embedded with a cryostat (Leica, Wentzler, Germany) were sliced so that the thickness of the sample was 8 μm, applied to a prepared specimen, then air-dried, and stored at −80 °C.

Immunostaining with Frozen Section of Mouse Tissue

The produced frozen section was fixed with acetone at 4 °C for 20 minutes, dried, and then washed with PBS. Subsequently, in order to block the tissue specimen, the mixture was washed with PBS, 5% goat serum/PBS was added, and the mixture was allowed to stand at room temperature for 30 minutes. In order to block the endogenous IgG, anti-mouse IgG Fab fragment (0.1 mg/ml in PBS) was added, and the mixture was allowed to stand at room temperature for 1 hour. An LSMEM2 monoclonal antibody diluted 2000 times with 5% goat serum/PBS was added, and the mixture was placed in a wet box and allowed to stand at 4 °C over night. Thereafter, in order to perform a secondary antibody reaction, the specimen was washed with PBS, the secondary antibody was then diluted 500 times, Hoechst33342 for staining cell nuclei was added, the mixture was allowed to stand at room temperature for 45 minutes, and observation was performed with a fluorescence microscope. As a result, it was confirmed that the protein disappeared in the LSMEM2 knockout mice (FIG. 4B).

Morphological observations of mouse heart tissue sections by hematoxylin eosin staining revealed no apparent phenotypes in the LSMEM2 knockout mice (FIG. 4C).

### Example 5

Crossbreeding Experiment with Laminopathy Model Mice LMNA H222P knock-in (KI) mice (Hum Mol Genet. 2005; 14(1): 155) were used as laminopathy model mice. LMNA H222P KI/KI, LSMEM2−/−mice were produced by crossbreeding the LMNA H222P KI mice with LSMEM2 KO mice. Analysis of survival periods, cardiac function analysis, and lower limb muscle strength measurement of LMNA H222P KI/KI, LSMEM2+/+mice and LMNA H222P KI/KI, LSMEM2−/−mice were performed. Cardiac function analysis was performed at the ages of 3, 6, and 9 months using Vevo 3100 imaging system to evaluate LVEF (left ventricular ejection fraction). The number of individuals used is as shown in the figure. Lower limb muscle strength measurement was performed at the age of 7 months. Lower limb muscle strength measurement was performed at the age of 29 weeks using 1300A 3-in-1 Whole Animal Muscle System (Aurora Scientific), and Twitch force, Tetanic force, and Force-frequency of gastrocnemius muscle were evaluated. For the analysis, DMA software (Aurora Scientific) was used. The comparison of the two groups was statistically examined by t-test.

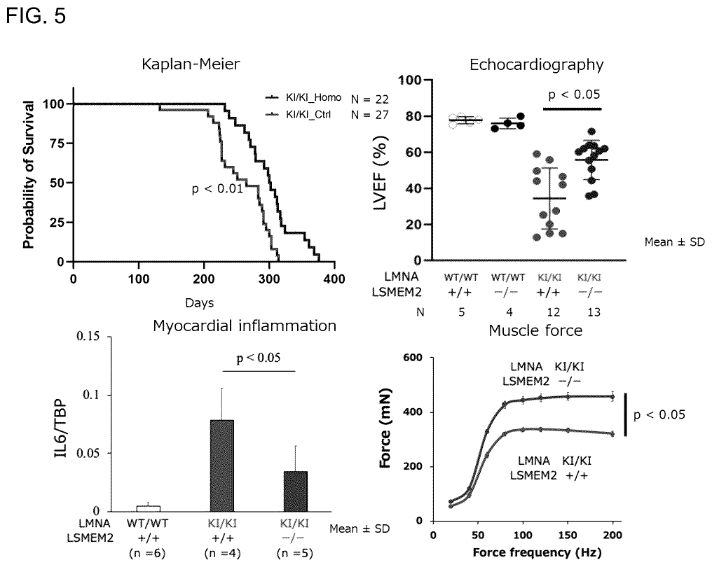

As a result, it was also shown that LSMEM2 KO mice improve all of the survival rate, cardiac function, cardiac inflammation signal, and skeletal muscle strength of the laminopathy model (LMNA H222P KI/KI) (FIG. 5).

### Example 6

### Production of AAV Vectors

MyoAAV2 vectors (Tabebordbar M. et al. Cell 2021) were produced by co-transfecting HEK293T cells with the transfer plasmid, Rep-cap plasmid, and helper plasmid, and viral particles were concentrated and purified by cesium chloride density gradient ultracentrifugation. MyoAAV2 U6-shRNA (LSMEM2) was produced to perform knockdown of skeletal muscle cell-specific LSMEM2. The shRNA designed shLSMEM2 (5′-CTCTCACTGATGGCTTCATTT-3′ (SEQ ID NO: 11) targeting mouse LSMEM2. As a control, shControl (5′-CCTAAGGTTAAGTCGCCCTCG-3′ (SEQ ID NO: 12)) targeting no mouse gene was designed.

### AAV Administration Experiment

MyoAAV2 U6-shRNA (LSMEM2) (hereinafter, also referred to as shLSMEM2) and MyoAAV2 shControl were intravenously injected at 7×10¹¹ genome copy (GC) from the orbital venous plexus of 10 week-old LMNA KI mice to evaluate the cardiac function.

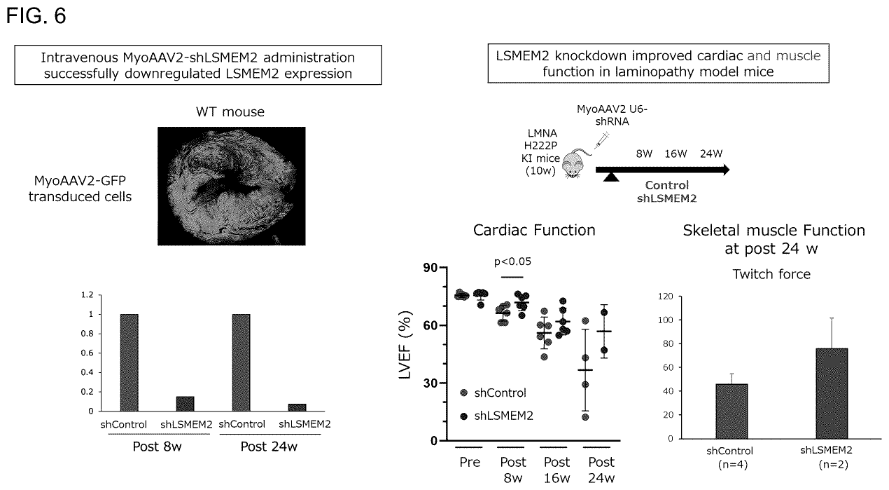

As a result, in the MyoAAV2 shControl administration group, a decrease in cardiac function over time was observed at the time points of 8 weeks, 16 weeks, and 24 weeks after administration, but in the shLSMEM2 administration group, this decrease in cardiac function was suppressed (FIG. 6, Cardiac Function). In the skeletal muscle function at Week 24 of administration, an improvement in twitch force was observed in the shLSMEM2 administration group (FIG. 6, Skeletal muscle Function at post 24 W). That is, it was shown that the postnatal LSMEM2 expression inhibition has a therapeutic effect in the laminopathy model.

### Example 7

### Mouse Aortic Arch Coarctation Model

Aortic arches of wild-type mice and LSMEM2 knockout mice were ligated, and a model in which a high pressure load was applied to the left ventricle was prepared. This model is very common as a pressure-loaded heart failure model used in the study of the circulatory area. A study using cardiac tissue for 1 week after surgery and evaluation of cardiac function by temporal echocardiographic observation for 1 and 4 weeks after surgery were performed.

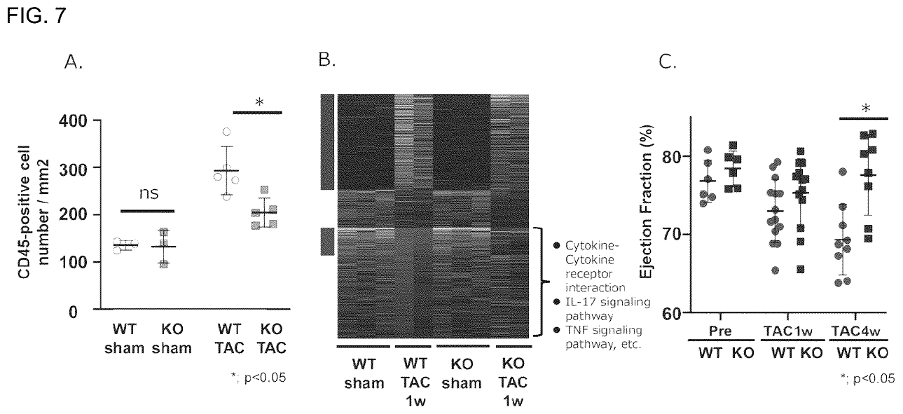

A cardiac tissue 1 week after surgery was extracted, and immunostaining was performed using a CD45 antibody which is a surface marker of mouse white blood cells by the same method as in FIG. 4 to examine infiltration of inflammatory cells into myocardial tissue. Significant intramyocardial inflammatory cell infiltration was recognized for 1 week after surgery in the wild-type mice, whereas it was significantly suppressed in the LSMEM2 knockout mice (FIG. 7A). RNA-seq analysis was performed by the same method as in FIG. 2 using a cardiac tissue for 1 week after surgery. In the wild-type mice, a significant increase in expression of the inflammation-related gene group was recognized for 1 week after surgery, consistent with the results of CD45 staining, whereas in the LSMEM2 knockout mice, the increase in expression was significantly suppressed (FIG. 7B). As a result of evaluating the cardiac function by echocardiography at 1 and 4 weeks after surgery, a decrease in cardiac function over time was observed in the wild-type mice, but this decrease in cardiac function was suppressed in the LSMEM2 knockout mice (FIG. 7C).

### INDUSTRIAL APPLICABILITY

According to the present invention, a mechanosignaling inhibitor containing a substance inhibiting the expression or function of LSMEM2 can be provided, so that it is possible to effectively treat cardiolaminopathy, severe heart failure due to other etiologies, and laminopathies.

This application is based on Japanese Patent Application No. 2022-136279 (filing date: Aug. 29, 2022), the entire contents of which are incorporated herein by reference.

1.-9. (canceled)

10. A method of inhibiting mechanosignaling in a subject, the method including administering to the subject a substance inhibiting the expression or function of LSMEM2.

11. The method according to claim 10, in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.

12. The method according to claim 10, in which the substance inhibiting the function of LSMEM2 is an antibody, degrader, or aptamer against LSMEM2.

13. The method according to claim 10, in which the substance inhibiting the expression or function of LSMEM2 is provided in a form of one or more expression vectors encoding the substance.

14. A method of inhibiting inflammation in a subject, the method including administering to the subject a substance inhibiting the expression or function of LSMEM2.

15. The method according to claim 14, in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.

16. The method according to claim 14, in which the substance inhibiting the function of LSMEM2 is an antibody, degrader, or aptamer against LSMEM2.

17. The method according to claim 14, in which the substance inhibiting the expression or function of LSMEM2 is provided in a form of one or more expression vectors encoding the substance.

18. A method of preventing and/or treating heart diseases in a subject, the method including administering to the subject a substance inhibiting the expression or function of LSMEM2.

19. The method according to claim 18, in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.

20. The method according to claim 18, in which the substance inhibiting the function of LSMEM2 is an antibody, degrader, or aptamer against LSMEM2.

21. The method according to claim 18, in which the substance inhibiting the expression or function of LSMEM2 is provided in a form of one or more expression vectors encoding the substance.

22. The method according to claim 18, in which the heart diseases include dilated cardiomyopathy.

23. A method for inhibiting abnormal vesicle trafficking in a subject, the method including administering to the subject a substance inhibiting the expression or function of LSMEM2.

24. The method according to claim 23, in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.

25. The method according to claim 23, in which the substance inhibiting the function of LSMEM2 is an antibody, degrader, or aptamer against LSMEM2.

26. The method according to claim 23, in which the substance inhibiting the expression or function of LSMEM2 is provided in a form of one or more expression vectors encoding the substance.

27. A method of preventing and/or treating laminopathies, the method including administering to a subject a substance inhibiting the expression or function of LSMEM2.

28. The method according to claim 27, in which the substance inhibiting the expression of LSMEM2 is
(a) a nucleic acid having RNAi activity against a transcription product of an LSMEM2 gene or a precursor thereof,
(b) an antisense nucleic acid to a transcription product of an LSMEM2 gene,
(c) a ribozyme nucleic acid to a transcription product of an LSMEM2 gene, or
(d) a nucleic acid sequence-recognizing module specifically binding to a target nucleotide sequence in an LSMEM2 gene.

29. The method according to claim 27, in which the substance inhibiting the function of LSMEM2 is an antibody, degrader, or aptamer against LSMEM2.

30. The method according to claim 27, in which the substance inhibiting the expression or function of LSMEM2 is provided in a form of one or more expression vectors encoding the substance.

31. The method according to claim 27, in which the laminopathies are selected from muscular dystrophy, cardiolaminopathy, lipodystrophy, or leukodystrophy.


SEQUENCE LISTING (12 sequences)

SEQ ID NO: 1 - RNA, 1434 nt, Homo sapiens
  source 1..1434: mol_type = mRNA
  CDS 28..522: protein_id = 2; translation = MPSLAPDCPLLAMPEETQEDSVAPMMPSQRSRGPLAPNHVHEVC LHQVESISDLHSGAGTLRPYLTEEARPWDELLGVLPPSLCAQAGCSPVYRRGGFLLLL ALLVLTCLVLALLAVYLSVLQSESLRILAHTLRTQEETLLKLRLASLSQLRRLNSSEA QAPS
gctgcatttgtccagtcctgctactggatgccatcattggcccccgactgcccactgcttgccatgcctgaggagacccaagaagactccgtggcgccaatgatgcccagccagaggagcagggggccattggcccccaaccacgtgcatgaggtatgcctgcaccaggtggagtccatcagcgacctacatagtggagcaggcacactgcgcccctatctaactgaagaggcacgaccgtgggatgagctgctgggcgttttgccgccgtcactgtgtgcccaggctggctgcagccctgtgtacagacgaggagggttcctgctgctgctcgcgctgctggtgctcacttgcctagtgctcgcactcctggctgtctacctgagcgtgctgcagagtgaatccctgcgcatcctggcacacacgctccgcacgcaggaggagacactactcaaactccgcttggccagcctcagccagcttcggaggctcaactccagtgaggcccaagcacccagctgagatgccatttggatctggggcctgggggtgtgtgttggtgggggaataaagtggctatgggcaggtctctccttccctactgctggctgccacatctacactatttccttggtgagatttttgtacaagaacctgttgttaacttaatggctgcctccctctcctgatctcttcaagccaggcctagccaagcctctggtgccaaagcctcgctttgggtggcccaaggtcaggggaaggggcaccagcctcctggctcctcctgtggcctgtcaacactctctggccaccccagatggcaggttctgaagtctagaaatagctgcccccatagcaatcacccaaactctgccagggctcccagtgagggtccagcctcagctcttaaagacaggctgggctctgagtagggggagaggcaccacctggggaagactgccttggagggcaaaggtgacatcgtggaagaccagttgggcccaagcctgggatagtagatggggagggagaacagaaaaatacccaggggttaagaccacagaaggctgaccgttctgtttaattatctgtaataatccttaaaaatcagcacacaaatccattccaggtagctttgccaccccacctgctgtggtggcatttgtccagatgccaccacccccctaagagtgggtcatcctgggggagcaaggcctgagaaaggaaaggaagggaaaggccccctagtgcctgccccacagccctgaggaaggcacatatttagcctggcctgagacaggacaggaaggggccacagggctgagtgcagggacaaaggggtcagggtcccaagtgtccgggcccccagctacccaccccatgtctgtttttggttttgtcattaaaaaaaataaagtgacaaatactggtgga

SEQ ID NO: 2 - protein, 164 aa, Homo sapiens
  TRANSMEM 100..122
  CARBOHYD 156: note = N-linked glycosylation
MPSLAPDCPLLAMPEETQEDSVAPMMPSQRSRGPLAPNHVHEVCLHQVESISDLHSGAGTLRPYLTEEARPWDELLGVLPPSLCAQAGCSPVYRRGGFLLLLALLVLTCLVLALLAVYLSVLQSESLRILAHTLRTQEETLLKLRLASLSQLRRLNSSEAQAPS

SEQ ID NO: 3 - RNA, 1431 nt, Homo sapiens
  source 1..1431: mol_type = mRNA
  CDS 28..519: protein_id = 4; translation = MPSLAPDCPLLAMPEETQEDSVAPMMPSQRSRGPLAPNHVHEVC LHQVESISDLHSGGTLRPYLTEEARPWDELLGVLPPSLCAQAGCSPVYRRGGFLLLLA LLVLTCLVLALLAVYLSVLQSESLRILAHTLRTQEETLLKLRLASLSQLRRLNSSEAQ APS
gctgcatttgtccagtcctgctactggatgccatcattggcccccgactgcccactgcttgccatgcctgaggagacccaagaagactccgtggcgccaatgatgcccagccagaggagcagggggccattggcccccaaccacgtgcatgaggtatgcctgcaccaggtggagtccatcagcgacctacatagtggaggcacactgcgcccctatctaactgaagaggcacgaccgtgggatgagctgctgggcgttttgccgccgtcactgtgtgcccaggctggctgcagccctgtgtacagacgaggagggttcctgctgctgctcgcgctgctggtgctcacttgcctagtgctcgcactcctggctgtctacctgagcgtgctgcagagtgaatccctgcgcatcctggcacacacgctccgcacgcaggaggagacactactcaaactccgcttggccagcctcagccagcttcggaggctcaactccagtgaggcccaagcacccagctgagatgccatttggatctggggcctgggggtgtgtgttggtgggggaataaagtggctatgggcaggtctctccttccctactgctggctgccacatctacactatttccttggtgagatttttgtacaagaacctgttgttaacttaatggctgcctccctctcctgatctcttcaagccaggcctagccaagcctctggtgccaaagcctcgctttgggtggcccaaggtcaggggaaggggcaccagcctcctggctcctcctgtggcctgtcaacactctctggccaccccagatggcaggttctgaagtctagaaatagctgcccccatagcaatcacccaaactctgccagggctcccagtgagggtccagcctcagctcttaaagacaggctgggctctgagtagggggagaggcaccacctggggaagactgccttggagggcaaaggtgacatcgtggaagaccagttgggcccaagcctgggatagtagatggggagggagaacagaaaaatacccaggggttaagaccacagaaggctgaccgttctgtttaattatctgtaataatccttaaaaatcagcacacaaatccattccaggtagctttgccaccccacctgctgtggtggcatttgtccagatgccaccacccccctaagagtgggtcatcctgggggagcaaggcctgagaaaggaaaggaagggaaaggccccctagtgcctgccccacagccctgaggaaggcacatatttagcctggcctgagacaggacaggaaggggccacagggctgagtgcagggacaaaggggtcagggtcccaagtgtccgggcccccagctacccaccccatgtctgtttttggttttgtcattaaaaaaaataaagtgacaaatactggtgga

SEQ ID NO: 4 - protein, 163 aa, Homo sapiens
  CARBOHYD 155: note = N-linked glycosylation
  TRANSMEM 99..120
MPSLAPDCPLLAMPEETQEDSVAPMMPSQRSRGPLAPNHVHEVCLHQVESISDLHSGGTLRPYLTEEARPWDELLGVLPPSLCAQAGCSPVYRRGGFLLLLALLVLTCLVLALLAVYLSVLQSESLRILAHTLRTQEETLLKLRLASLSQLRRLNSSEAQAPS

SEQ ID NO: 5 - RNA, 23 nt, synthetic construct
  source 1..23: mol_type = other RNA
actggctcctaaccacgtgcagg

SEQ ID NO: 6 - RNA, 83 nt, synthetic construct
  source 1..83: mol_type = other RNA
gttttagagctagaaatagcaagttaaaataaggctagtccgttatcaacttgaaaaagtggcaccgagtcggtggtgctttt

SEQ ID NO: 7 - DNA, 25 bp, synthetic construct
  source 1..25: PCR_primers = fwd_name:Mouse_RT_PCR_L, fwd_seq:agcttctgggtgtcttaccaccatc, rev_name:Mouse_RT_PCR_R, rev_seq:caatttgagcagagtctcctcttgtgtgc,; mol_type = other DNA
agcttctgggtgtcttaccaccatc

SEQ ID NO: 8 - DNA, 29 bp, synthetic construct
  source 1..29: mol_type = other DNA; note = Mouse_RT-PCR_R
caatttgagcagagtctcctcttgtgtgc

SEQ ID NO: 9 - DNA, 21 bp, synthetic construct
  misc_feature 1..19: note = RNA
  misc_feature 20..21: note = DNA
  source 1..21: mol_type = other DNA
gcacagtggaggctcactatt

SEQ ID NO: 10 - RNA, 21 nt, synthetic construct
  source 1..21: mol_type = other RNA
tagtgagcctccactgtgcag

SEQ ID NO: 11 - DNA, 21 bp, synthetic construct
  source 1..21: mol_type = other DNA; note = shLSMEM2
ctctcactgatggcttcattt

SEQ ID NO: 12 - DNA, 21 bp, synthetic construct
  source 1..21: mol_type = other DNA; note = shControl
cctaaggttaagtcgccctcg